(19) United States
(12) Patent Application Publication
(10) Pub. No.: US 2022/0305031 A1
(43) Pub. Date: Sep. 29, 2022

(54) METHODS FOR REDUCING TUMOR PROGRESSION AND FIBROSIS AND INCREASING ADAPTIVE IMMUNITY IN MALIGNANCIES

(71) Applicant: GEORGETOWN UNIVERSITY, Washington, DC (US)

(72) Inventors: Robert I. GLAZER, Potomac, MD (US); Moshe LEVI, Washington, DC (US); Hongyan YUAN, Damascus, MD (US); Suman RANJIT, Arlington, VA (US)

(21) Appl. No.: 17/618,302

(22) PCT Filed: Jun. 11, 2020
(86) PCT No.: PCT/US2020/037287

§ 371 Date: Dec. 10, 2021

Related U.S. Application Data
(60) Provisional application No. 62/860,561, filed on Jun. 12, 2019.

(51) Int. Cl. A61K 31/575; A61K 31/337; A61K 31/704; A61K 31/513; A61K 31/675; A61K 33/243; A61K 31/282; A61K 31/395; A61K 31/7068; A61K 31/475; A61P 35/00

(52) U.S. Cl. CPC A61K 31/575; A61K 31/337; A61K 31/704; A61K 31/513; A61K 31/675; A61K 33/243; A61K 31/282; A61K 31/5545; A61K 31/7068; A61K 31/475; A61P 35/00

(57) ABSTRACT

Aspects of the technology described herein relate to a method of treating a malignancy in a subject. This method involves selecting a subject having a malignancy and administering dimethyl-3-beta-hydroxy-cholenamide (DMHCA) or derivative thereof to the subject in an amount effective to treat the malignancy. Methods of reducing malignancy-associated fibrosis in a subject and pharmaceutical combinations comprising (i) DMHCA or derivative thereof and (ii) one or more immune checkpoint inhibitors are also disclosed.

This application claims the priority benefit of U.S. Provisional Patent Application Ser. No. 62/860,561, filed Jun. 12, 2019, which is hereby incorporated by reference in its entirety.

This invention was made with government support under grant numbers R01AG049493 and R01DK116567 awarded by National Institutes of Health. The government has certain rights in the invention.

### FIELD

Aspects of the technology described herein relate to methods of treating a malignancy in a subject, methods of reducing malignancy-associated fibrosis in a subject, and pharmaceutical combinations of dimethyl-3-beta-hydroxy-cholenamide (DMHCA) and one or more immune checkpoint inhibitors.

### BACKGROUND

Cancer progression is influenced by several factors, including tumor heterogeneity, the tumor microenvironment (TME), and immune suppression. During the transition from pre-invasive to invasive ductal breast cancer, the TME undergoes extensive changes in gene expression accompanied by extracellular matrix (ECM) remodeling and an altered immune response (Ma et al., “Gene Expression Profiling of the Tumor Microenvironment During Breast Cancer Progression,” Breast Cancer Res. 11(1):R7 (2009)), as well as a stromal-derived gene expression signature that relates to poor outcome in multiple breast cancer subtypes (Finak et al., “Stromal Gene Expression Predicts Clinical Outcome in Breast Cancer,” Nat. Med. 14(5):518-527 (2008)). In hormone receptor-negative breast cancer, the repertoire of stromal cell types in the TME (Hanahan & Coussens, “Accessories to the Crime: Functions of Cells Recruited to the Tumor Microenvironment,” Cancer Cell 21(3):309-322 (2012); Bhowmick et al., “Stromal Fibroblasts in Cancer Initiation and Progression,” Nature 432(7015):332-337 (2004); Kalluri & Zeisberg, “Fibroblasts in Cancer,” Nat. Rev. Cancer 6(5):392-401 (2006)) leads to large fibrotic foci and early metastasis (Van den Eynden et al., “A Fibrotic Focus Is a Prognostic Factor and a Surrogate Marker for Hypoxia and (Lymph)Angiogenesis in Breast Cancer: Review of the Literature and Proposal on the Criteria of Evaluation,” Histopathology 51(4):440-451 (2007)). Fibrosis is associated with the secretion of chemokines, cytokines, growth factors, and collagen by cancer-associated fibroblasts (Kalluri & Zeisberg, “Fibroblasts in Cancer,” Nat. Rev. Cancer 6(5):392-401 (2006); Harper & Sainson, “Regulation of the Anti-Tumour Immune Response by Cancer-Associated Fibroblasts,” Semin. Cancer Biol. 25:69-77 (2014)), which collectively promote angiogenesis, tumor growth, and invasion (Orimo et al., “Stromal Fibroblasts Present in Invasive Human Breast Carcinomas Promote Tumor Growth and Angiogenesis Through Elevated SDF-1/CXCL12 Secretion,” Cell 121(3):335-348 (2005); Feig et al., “Targeting CXCL12from FAP-Expressing Carcinoma-Associated Fibroblasts Synergizes with anti-PD-L1 Immunotherapy in Pancreatic Cancer,” Proc. Natl. Acad. Sci. USA 110(50):20212-20217 (2013)). This process poses a major risk factor for the development of precancerous lesions (Boyd et al., “Breast Tissue Composition and Susceptibility to Breast Cancer,” J. Natl. Cancer Inst. 102(16):1224-1237 (2010)), and invasive and metastatic disease (Van den Eynden et al., “A Fibrotic Focus Is a Prognostic Factor and a Surrogate Marker for Hypoxia and (Lymph)Angiogenesis in Breast Cancer: Review of the Literature and Proposal on the Criteria of Evaluation,” Histopathology 51(4):440-451 (2007); Gill et al., “The Association of Mammographic Density with Ductal Carcinoma in Situ of the Breast: The Multiethnic Cohort,” Breast Cancer Res. 8(3):R30 (2006); Hasebe, “Tumor-Stromal Interactions in Breast Tumor Progression-Significance of Histological Heterogeneity of Tumor-Stromal Fibroblasts,” Expert Opin. Ther. Targets 17(4):449-460 (2013); Malik et al., “Underestimation of Malignancy in Biopsy-Proven Cases of Stromal Fibrosis,” Br. J. Radiol. 87(1039):20140182 (2014); Ao et al., “Identification of Cancer-Associated Fibroblasts in Circulating Blood from Patients with Metastatic Breast Cancer,” Cancer Res. 75(22):4681-4687 (2015)), and results in epithelial to mesenchymal transition (EMT), a common feature of advanced disease independently of hormone or HER2 receptor status (Bayraktar et al., “Histopathological Features of Non-Neoplastic Breast Parenchyma Do Not Predict BRCA Mutation Status of Patients with Invasive Breast Cancer,” Biomark. Cancer 7:39-49 (2015)) and increased metastatic potential (Chaffer & Weinberg, “A Perspective on Cancer Cell Metastasis,” Science 331(6024):1559-1564 (2011); Dunlap et al., “Dietary Energy Balance Modulates Epithelial-to-Mesenchymal Transition and Tumor Progression in Murine Claudin-Low and Basal-Like Mammary Tumor Models,” Cancer Prev. Res. 5(7):930-942 (2012); Tam & Weinberg, “The Epigenetics of Epithelial-Mesenchymal Plasticity in Cancer,” Nat. Med. 19:1438-1449 (2013)). Fibrosis also sets into motion processes that result in immune suppression (Hanahan & Coussens, “Accessories to the Crime: Functions of Cells Recruited to the Tumor Microenvironment,” Cancer Cell 21:309-322 (2012); Korkaya et al., “Breast Cancer Stem Cells, Cytokine Networks, and the Tumor Microenvironment,” J. Clin. Invest. 121:3804-3809 (2011)) through secretion of a dense fibrotic collagen matrix that impedes CD8⁺ effector T cell penetration into the tumor bed (Gotwals et al., “Prospects for Combining Targeted and Conventional Cancer Therapy with Immunotherapy,” Nat. Rev. Cancer 17:286-301 (2017) and by inflammatory factors within the TME (Li et al., “Infiltration of CD8(+) T Cells into Tumor Cell Clusters in Triple-Negative Breast Cancer,” Proc. Natl. Acad. Sci. USA 116:3678-3687 (2019)). In fibrotic tissue, secretion of IL1, IL6, TNFα, CXCL1, CCL5 and other proinflammatory factors (Joyce & Pollard, “Microenvironmental Regulation of Metastasis,” Nat. Rev. Cancer 9(4):239-252 (2009); Grivennikov et al., “Immunity, Inflammation, and Cancer,” Cell 140(6):883-899 (2010)) facilitate immune suppression (Park & Scherer, “Leptin and Cancer: from Cancer Stem Cells to Metastasis,” Endocr. Relat. Cancer 18(4):C25-C29 (2011); Zheng et al., “Leptin Deficiency Suppresses MMTV-Wnt-1 Mammary Tumor Growth in Obese Mice and Abrogates Tumor Initiating Cell Survival,” Endocr. Relat. Cancer 18(4):491-503 (2011)) by recruitment and activation of regulatory T cells (Treg), myeloid-derived suppressor cells (MDSC) and tumor-activated macrophages that collectively inhibit CD8+ cytotoxic effector T cell activation and antigen presentation (Mellman et al., “Cancer Immunotherapy Comes of Age,” Nature 480(7378):480-489 (2011); Pardoll, “The Blockade of Immune Checkpoints in Cancer Immunotherapy,” Nat. Rev. Cancer 12(4):252-264 (2012)). Chemokines, including CXCL1, denote poor survival in breast cancer subjects (Zou et al., “Elevated CXCL1 Expression in Breast Cancer Stroma Predicts Poor Prognosis and Is Inversely Associated with Expression of TGF-Beta Signaling Proteins,” BMC Cancer 14:781 (2014)), promote MDSC infiltration and metastasis in triple-negative MDA-MB-231 xenografts (Acharyya et al., “A CXCL1 Paracrine Network Links Cancer Chemoresistance and Metastasis,” Cell 150(1):165-178 (2012)) and block adaptive immunity in the syngeneic E0771 mammary tumor model (Yuan et al., “Plac1 Is a Key Regulator of the Inflammatory Response and Immune Tolerance In Mammary Tumorigenesis,” Sci. Rep. 8(1):5717 (2018)). Importantly, the CXCL1/CXCR2 axis is a dominant feature in both NeuT/ATTAC (Yuan et al., “MMTV-NeuT/ATTAC Mice: A New Model for Studying the Stromal Tumor Microenvironment,” Oncotarget. 9(8):8042-8053 (2018)) and PPARd/ATTAC (Yuan et al., “PPARdelta Induces Estrogen Receptor-Positive Mammary Neoplasia Through an Inflammatory and Metabolic Phenotype Linked to mTOR Activation,” Cancer Res. 73(14):4349-4361 (2013)) mice, and its disruption inhibits MDSC activation and enhances the efficacy of anti-PD-1 therapy (Highfill et al., “Disruption of CXCR2-Mediated MDSC Tumor Trafficking Enhances Anti-PD1 Efficacy,” Sci. Transl. Med. 6(237):237ra67 (2014)). Other markers of fibroblast activation include fibroblast activation protein (FAP), a cell surface serine dipeptidase involved in ECM remodeling, inflammation, and tumor growth (Cheng & Weiner, “Tumors and Their Microenvironments: Tilling the Soil. Commentary Re: A. M Scott et al., ‘A Phase I Dose-Escalation Study of Sibrotuzumab in Patients with Advanced or Metastatic Fibroblast Activation Protein-Positive Cancer,’ Clin. Cancer Res. 9:1639-1647 (2003),” Clin. Cancer Res. 9:1590-1595 (2003); Zi et al., “Fibroblast Activation Protein Alpha in Tumor Microenvironment: Recent Progression and Implications (Review),” Mol. Med. Rep. 11(5):3203-3211 (2015)). FAP is highly expressed in the stroma and in tumor-activated macrophages in all breast cancer subtypes (Tchou et al., “Fibroblast Activation Protein Expression by Stromal Cells and Tumor-Associated Macrophages in Human Breast Cancer,” Hum. Pathol. 44:2549-2557 (2013)), and denotes increased invasion (Park et al., “Expression of Cancer-Associated Fibroblast-Related Proteins Differs Between Invasive Lobular Carcinoma and Invasive Ductal Carcinoma,” Breast Cancer Res. Treat. 159(1):55-69 (2016)) and microinvasion in DCIS (Hua et al., “Expression and Role of Fibroblast Activation Protein-Alpha in Microinvasive Breast Carcinoma,” Diagn. Pathol. 6:111 (2011)), and poor survival (Jia et al., “FAP-Alpha (Fibroblast Activation Protein-Alpha) Is Involved in the Control of Human Breast Cancer Cell Line Growth and Motility via the FAK Pathway,” BMC Cell Biol. 15:16 (2014)). Interestingly, FAP⁺ stromal cells served as an effective vaccine in a syngeneic model of triple-negative breast cancer (TNBC) that reduced fibrosis and tumor growth (Meng et al., “Immunization of Stromal Cell Targeting Fibroblast Activation Protein Providing Immunotherapy to Breast Cancer Mouse Model,” Tumour. Biol. 37:10317-10327 (2016)).

One of the central challenges in cancer treatment is the identification of factors within the TME that increase tumor progression and prevent the immune system from eradicating the tumor. The cell-centric hallmarks of cancer originally proposed (Hanahan & Weinberg, “The Hallmarks of Cancer,” Cell 100:57-70 (2000)) are exceedingly more complex, and must now take into account the multi-faceted role of stromal cells within the TME (Polyak et al., “Co-Evolution of Tumor Cells and Their Microenvironment,” Trends Genet. 25:30-38 (2009); Pietras & Ostman, “Hallmarks of Cancer: Interactions With the Tumor Stroma,” Exp. Cell Res. 316:1324-1331 (2010); Hanahan & Weinberg, “Hallmarks of Cancer: The Next Generation,” Cell 144:646-674 (2011); Hanahan & Coussens, “Accessories to the Crime: Functions of Cells Recruited to the Tumor Microenvironment,” Cancer Cell 21:309-322 (2012)). Although the TME is emerging as an important determinant of tumorigenesis, as well as an attractive target for therapy (Tchou & Conejo-Garcia, “Targeting the Tumor Stroma as a Novel Treatment Strategy for Breast Cancer: Shifting From the Neoplastic Cell-Centric to a Stroma-Centric Paradigm,” Adv. Pharmacol. 65:45-61 (2012)), identifying the immune, metabolic, and signaling pathways that orchestrate the symbiotic relationship between tumor and stromal tissue remains one of the primary challenges for developing new cancer therapies.

The present disclosure is directed to overcoming these and other deficiencies in the art.

### SUMMARY

One aspect of the technology described herein relates to a method of treating a malignancy in a subject. This method involves selecting a subject having a malignancy and administering dimethyl-3-beta-hydroxy-cholenamide (DMHCA) or a derivative thereof to the subject in an amount effective to treat the malignancy.

Another aspect of the technology described herein relates to a method of reducing malignancy-associated fibrosis in a subject. This method involves selecting a subject having a malignancy and administering DMHCA or a derivative thereof to the subject in an amount effective to reduce malignancy-associated fibrosis in the subject.

A further aspect of the technology described herein relates to a pharmaceutical combination. This combination comprises: (i) DMHCA or a derivative thereof and (ii) one or more immune checkpoint inhibitors.

As described herein, the development of fibrosis is a requisite factor for tumor progression, altering the inflammatory and immunomodulatory environment in the TME. Among other benefits, therapeutic targeting of fibrosis using DMHCA or a derivative thereof provides a unique therapeutic modality that can be administered to reduce tumor associated fibrosis and enhance the anti-tumor immune response in a patient, thereby enhancing treatment outcomes and survival.

### BRIEF DESCRIPTION OF THE DRAWINGS

FIG. 1 is a schematic illustration showing adipocyte-specific expression of the caspase 8-FKBP transgene in Fat Apoptosis Through Targeted Activation of Caspase 8 (FAT-ATTAC) mice. FAT-ATTAC mice express a myristoylated-FKBPv-caspase 8 fusion protein under the control of the adipose-targeted minimal Fabp4 promoter (Pajvani et al., “Fat Apoptosis Through Targeted Activation of Caspase 8: A New Mouse Model of Inducible and Reversible Lipoatrophy,” Nat. Med. 11(7):797-803 (2005), which is hereby incorporated by reference in its entirety). The linearized construct (top) shows the myristoylated-FKBPv-caspase 8 fusion protein under the control of the adipose-specific minimal Fabp4 promoter. As shown in the bottom left panel, caspase is activated by dimerization of adjacent FKBPv domains (crescents) by AP21087 (circles), which results in adipose tissue ablation and its replacement by fibrotic tissue. The bottom right panel shows the approach taken to produce NeuT/ATTAC mice. (Figure adapted from Pajvani et al., “Fat Apoptosis through Targeted Activation of Caspase 8: A New Mouse Model of Inducible and Reversible Lipoatrophy,” Nat. Med. 11(7):797-803 (2005), which is hereby incorporated by reference in its entirety.)

FIG. 2 shows that conditional induction of fibrosis by treatment with AP21087 (AP) increased tumor proliferation and fibrosis in PPARd/ATTAC and NeuT/ATTAC mice. Mice at 6 weeks of age (w.o.a.) were injected i.p. with vehicle (PPARd/ATTAC or NeuT/ATTAC) or 0.4 mg/kg AP (+AP) 3 times per week until tumor appeared. Formalin fixed, paraffin embedded (FFPE) sections were stained with hematoxylin and eosin (H&E) stain, Masson's Trichrome stain and Picrosirius Red stain (collagen I/III), fibroblast activation protein (FAP), and smooth muscle actin (SMA). Ki67 and CD31 expression were also evaluated. Magnification 400×.

FIG. 3 are images showing fibrosis in six biopsies of HER2⁺ breast cancer. All biopsies expressed an abundance of FAP by immunohistochemistry (IHC) and collagen as determined by Picrosirius Red staining.

FIG. 4 is a table listing collagen (Col) gene expression in mammary tumors from NeuT/ATTAC, PPARd/ATTAC, and ATTAC mice after AP treatment. Shown are the major Col genes expressed in tumors from NeuT/ATTAC and PPARd/ATTAC mice or the mammary gland of ATTAC mice after AP treatment as determined with an Affymetrix genomic array. Col1a2 and Col3a1 were the most abundant Col genes in the fibrotic tissue of all mice. NeuT/ATTAC tumors expressed Col9a1 and Col11a1 that were not present in ATTAC or PPARd/ATTAC mice. PPARd/ATTAC tumors expressed Col5a2 and Col6a3 that were not present in NeuT/ATTAC tumors, but were present at similar levels in ATTAC mice.

FIGS. 5A-5B show tumor progression (FIG. 5A) and tumor multiplicity (FIG. 5B) in DMHCA-treated NeuT/ATTAC mice. Fibrosis was induced in NeuT/ATTAC mice by i.p. treatment with 0.4 mg/kg AP twice/week beginning at 6 w.o.a. Mice were maintained on a diet containing 0.05% DMHCA (100 mg/kg) beginning at 8 w.o.a. until tumors appeared (Days on AP). DMHCA delayed and reduced tumor progression (FIG. 5A) and markedly reduced tumor multiplicity (FIG. 5B). Control, N=6; DMHCA, N=7.

FIGS. 6A-6D show immune cell analysis following DMHCA treatment of NeuT/ATTAC mice. FIG. 6A shows representative fluorescence-activated cell sorting (FACS) profiles of tumor infiltrates and spleen from control and DMHCA-treated NeuT/ATTAC mice. DMHCA reduced regulatory T (Treg) cells, monocytic myeloid-derived suppressor cells (M-MDSCs), and granulocytic myeloid-derived suppressor cells (G-MDSCs) in tumor infiltrates (FIG. 6B), and markedly reduced Treg cells and G-MDSCs in the spleen (FIG. 6C). DMHCA also enhanced CD4⁺ and CD8⁺ effector T cells in both tissues (FIGS. 6B and 6C). FIG. 6D shows Treg cells graphed on an expanded scale. For each cell type in FIGS. 6B-6C, left bar=control (“Ctl”), right bar=DMHCA.

FIG. 7 are representative FFPE sections showing that DMHCA treatment reduces macrophage infiltration (left panel) and CXCL1 expression (right panel) in tumors from NeuT/ATTAC mice. Mice were treated as in FIG. 5. FFPE sections were stained for macrophage marker F4/80 and CXCL1. Magnification 400×.

FIGS. 8A-8G show the results of fluorescence life-time microscopy (FLIM) analysis and second harmonic imaging (SHG) of tumors from control and DMHCA-treated NeuT/ATTAC mice. The FLIM analysis shown in FIGS. 8A-8D indicates reduced collagen and increased free NADH in tumors from NeuT/ATTAC mice after DMHCA treatment. The phasor mapped image of the control tumor (FIG. 8A) differs markedly from the image of the DMHCA-treated tumor (FIG. 8B), that is indicative of tumor heterogeneity. Excessive collagen I deposition is present in the control tumor (FIG. 8C, central dark grey shading) and is largely absent in the DMHCA-treated tumor (FIG. 8D). The control tumor also exhibits more free NADH (FIG. 8C, outer dark grey shading), that indicates a glycolytic metabolism in comparison to more bound NADH and an oxidative metabolism in the DMHCA-treated tumor (FIG. 8D, grey shading). FIGS. 8E-8G are images of second harmonic generation (SHG) microscopy of tumors from control and DMHCA-treated mice. SHG is generated by the interaction of light with the non-centrosymmetric structure of collagen I fibers, and is indicative of fibrosis. FIG. 8E shows an SHG image of a control tumor showing extensive collagen I deposition. FIG. 8F shows an SHG image of a DMHCA-treated tumor showing that collagen is largely absent, indicating a marked reduction in fibrosis. The red shaded cursor (“a”) of the corresponding phasor plot (FIG. 8G) indicates a signal at s=0, g=1, since the harmonic generation signal is not delayed compared to fluorescence.

### DETAILED DESCRIPTION

In this specification and the appended claims, the singular forms “a”, “an”, and “the” include plural references unless the context clearly dictates otherwise.

The terms “comprising”, “comprises” and “comprised of” as used herein are synonymous with “including”, “includes” or “containing”, “contains”, and are inclusive or open-ended and do not exclude additional, non-recited members, elements, or method steps. The terms “comprising”, “comprises”, and “comprised of” also encompass the term “consisting of”.

The recitation of numerical ranges by endpoints includes all numbers and fractions subsumed within the respective ranges, as well as the recited endpoints.

One aspect of the present disclosure relates to a method of treating a malignancy in a subject. This method involves selecting a subject having a malignancy and administering dimethyl-3-beta-hydroxy-cholenamide (DMHCA) or a derivative thereof to the subject in an amount effective to treat the malignancy.

In accordance with this and all aspects of the present disclosure, the terms “malignancy” or “cancer” encompass conditions in which abnormal cells divide without control and can invade nearby tissues. Malignant cells can also spread to other parts of the body through the blood and lymph systems. As used herein, a “tumor” or “neoplasm” refers to the abnormal mass of tissue that results when these abnormal cells divide more than they should or do not die when they should.

There are several main types of malignancy, all of which can be treated in accordance with the various methods and compositions as described herein. In some embodiments, the malignancy is a “carcinoma”, which is a malignancy that begins in the skin or in tissues that line or cover internal organs. In other embodiments, the malignancy is a “sarcoma”, which is a malignancy that begins in bone, cartilage, fat, muscle, blood vessels, or other connective or supportive tissue. In other embodiments, the malignancy is a “leukemia” which is a malignancy that starts in blood-forming tissue, such as the bone marrow, and causes large numbers of abnormal blood cells to be produced and enter the blood. In other embodiments, the malignancy is a “lymphoma” or “multiple myeloma”, which are malignancies that begin in the cells of the immune system. In other embodiments, the malignancy is a central nervous system malignancy, which is a malignancy that begins in the tissues of the brain and spinal cord.

In some embodiments of the methods described herein, the malignancy is a breast malignancy. As used herein, a “breast malignancy” refers to a condition characterized by anomalous rapid proliferation of abnormal cells that originate in the breast of a subject. Malignant breast cells may be identified in one or both breasts only and not in another tissue or organ, in one or both breasts and one or more adjacent tissues or organs (e.g., lymph node), or in one or both breasts and one or more non-adjacent tissues or organs to which the breast malignancy cells have metastasized.

In some embodiments, the subject has a breast malignancy that is a ductal carcinoma in situ, invasive ductal carcinoma (e.g., tubular carcinoma, medullary carcinoma, mucinous carcinoma, papillary carcinoma, and cribriform carcinoma), invasive lobular carcinoma, inflammatory breast cancer, lobular carcinoma in situ, or metastatic breast cancer.

In some embodiments, the subject treated in accordance with the methods described herein has a breast malignancy characterized by its progesterone receptor and human epidermal growth factor receptor 2 (HER2) status. For example, the subject's breast malignancy may be a progesterone receptor positive (PR⁺) or progesterone receptor negative (PR⁻) malignancy. In another embodiment, the subject's breast malignancy is human epidermal growth factor receptor 2 positive (HER2⁺) or human epidermal growth factor receptor 2 negative (HER2⁻) malignancy. In another embodiment, the subject's breast malignancy is an androgen receptor positive (AR⁺) or androgen receptor negative (AR⁻) malignancy.

In some embodiments, the subject has a breast malignancy classified as an estrogen receptor positive (ER⁺) or an estrogen receptor negative (ER⁻) malignancy. ER⁺ breast malignancies are malignancies where active ER signaling drives proliferation. There are two major isoforms of estrogen receptor, ERα and ERβ. ERα and ERβ are encoded by two unique genes that reside on distinct chromosomes and each isoform is responsible for the regulation of a specific set of genes that elicit tissue-specific effects. The role of ERα in cancer initiation and progression has been well established in breast cancer (Fullwood et al., “An Oestrogen-Receptor-a-Bound Human Chromatin Interactome,” Nature 462:58-64 (2009); Sommer et al., “Estrogen Receptor and Breast Cancer,” Semin. Cancer Biol. 11:339-352 (2001); Oxelmark et al., “The Cochaperone p23 Differentially Regulates Estrogen Receptor Target Genes and Promotes Tumor Cell Adhesion and Invasion,” Mol. Cell. Biol. 26:5205-13 (2006); Simpson et al., “High Levels of Hsp90 Cochaperone p23 Promote Tumor Progression and Poor Prognosis in Breast Cancer by Increasing Lymph Node Metastases and Drug Resistance,” Cancer Res. 70:8446-56 (2010), each of which is hereby incorporated by reference in its entirety).

In some embodiments of the methods described herein, the subject has an HER2⁺/ER⁻ breast malignancy. The presence and/or absence of HER2 and ER (as well as other hormone receptors) in breast malignancies or malignant tumor cells can be readily evaluated, e.g., by immunohistochemistry (IHC). Certain embodiments of the methods disclosed herein further comprise determining that the tumor expresses HER2 and optionally one or more other receptors (e.g., PR, HER2, AR).

In some embodiments of the methods disclosed herein, the subject has a pancreatic malignancy. Pancreatic malignancies that can be treated in accordance with the methods herein include, but are not limited to, acinar cell carcinoma, adenocarcinoma (ductal adenocarcinoma), adenosquamous carcinoma, anaplastic carcinoma, cystadenocarcinoma, duct-cell carcinoma (ductal adrenocarcinoma), giant-cell carcinoma (osteoclastoid type), a giant cell tumor, intraductal papillary-mucinous neoplasm (IPMN), mixed-cell carcinoma, mucinous (colloid) carcinoma, mucinous cystadenocarcinoma, papillary adenocarcinoma, pleomorphic giant-cell carcinoma, serous cystadenocarcinoma, small-cell (oat-cell) carcinoma, solid tumors, and pseudopapillary tumors.

In some embodiments of the methods disclosed herein, the subject has a lung malignancy. Lung malignancies that can be treated in accordance with the methods herein include, but are not limited to, non-small cell lung cancer, small cell lung cancer, and lung carcinoid tumors.

In some embodiments of the methods disclosed herein, the subject has a liver malignancy. Liver malignancies that can be treated in accordance with the methods herein include, but are not limited to, hepatocellular carcinoma (e.g., fibrolamellar hepatocellular carcinoma), intrahepatic cholangiocarcinoma (bile duct malignancy), angiosarcoma, hemangiosarcoma, and hepatoblastoma.

In some embodiments of the methods disclosed herein, the subject has a gastrointestinal malignancy. Gastrointestinal malignancies that can be treated in accordance with the methods herein include, without limitation, an oral cavity malignancy, pharyngeal malignancy, esophageal malignancy, stomach (i.e., gastric) malignancy, small intestinal malignancy, cecal malignancy, colon malignancy, rectal malignancy, anal malignancy, salivary gland malignancy, liver malignancy, pancreatic malignancy, biliary malignancy (bile duct malignancy), gall bladder malignancy, or peritoneal malignancy.

In some embodiments of the methods disclosed herein, the subject has a stomach malignancy. Stomach malignancies that can be treated in accordance with the methods herein include, but are not limited to, adenocarcinoma (distal stomach cancer, proximal stomach cancer, diffuse stomach cancer), gastrointestinal stromal tumors, carcinoid tumors, lymphoma, squamous cell carcinoma, small cell carcinoma, leiomyosarcoma, signet ring cell carcinoma, gastric lymphoma (MALT lymphoma), and linitis plastica.

In some embodiments of the methods disclosed herein, the subject has a gall bladder malignancy. Gall bladder malignancies that can be treated in accordance with the methods herein include, but are not limited to, adenocarcinomas (papillary adenocarcinoma), adenosquamous carcinomas, squamous cell carcinomas, and carcinosarcomas.

In some embodiments of the methods disclosed herein, the subject has an esophageal malignancy. Esophageal malignancies that can be treated in accordance with the methods herein include, but are not limited to, adenocarcinoma, squamous cell carcinoma, small cell carcinoma, lymphoma, melanomas, and sarcoma.

In some embodiments of the methods disclosed herein, the subject has a colorectal malignancy. Colorectal malignancies that can be treated in accordance with the methods herein include malignancies that originate in the colon and rectum. Exemplary colon malignancies include, but are not limited to, adenocarcinoma, carcinoid tumors, gastrointestinal stromal tumors, lymphomas, and sarcomas. Exemplary rectal malignancies include, but are not limited to, adenocarcinoma, carcinoid tumors, gastrointestinal stromal tumors, lymphomas, and sarcomas.

In some embodiments of the methods disclosed herein, the subject has a renal malignancy. Renal malignancies that can be treated in accordance with the methods herein include, but are not limited to, clear renal cell carcinoma, papillary renal cell carcinoma, chromophobe renal carcinoma, collecting duct renal cell carcinoma, multiocular cystic renal cell carcinoma, medullary carcinoma, mucinous tubular and spindle cell carcinoma, neuroblastoma-associated renal cell carcinoma, and unclassified renal cell carcinoma.

In some embodiments of the methods disclosed herein, the subject has a bladder malignancy. Bladder malignancies that can be treated in accordance with the methods herein include, but are not limited to, urothelial carcinoma (transitional cell carcinoma), squamous cell carcinoma, adenocarcinoma, small cell carcinoma, and sarcoma.

In some embodiments of the methods disclosed herein, the subject has a prostate malignancy. Prostate malignancies that can be treated in accordance with the methods herein include, but are not limited to, adenocarcinoma, sarcoma, small cell carcinomas, neuroendocrine tumors (other than small cell carcinomas), and transitional cell carcinomas.

In some embodiments of the methods disclosed herein, the subject has a cervical malignancy. Cervical malignancies that can be treated in accordance with the methods herein include, but are not limited to, squamous cell carcinoma, adenocarcinoma, adenocarcinoma, melanoma, sarcoma, and lymphoma.

In some embodiments of the methods disclosed herein, the subject has a ovarian malignancy. Ovarian malignancies that can be treated in accordance with the methods herein include, but are not limited to, epithelial cell ovarian malignancy, germ cell ovarian malignancy, stromal cell ovarian malignancy, and small cell carcinoma.

In some embodiments of the methods disclosed herein, the subject has a testicular malignancy. Testicular malignancies that can be treated in accordance with the methods herein include, but are not limited to, classical seminoma, spermatocytic seminoma, embryonal carcinoma, yolk sac carcinoma, choriocarcinoma, teratoma, carcinoma in situ, leydig cell tumors, sertoli cell tumors, lymphoma, and leukemia.

In some embodiments of the methods disclosed herein, the subject has a skin malignancy. Skin malignancies that can be treated in accordance with the methods herein include, but are not limited to, basal cell carcinoma, squamous cell carcinoma, keratoacanthoma, melanoma, Merkel cell carcinoma, Kaposi sarcoma, cutaneous lymphoma, skin adnexal tumors, and sarcoma.

In some embodiments of the methods disclosed herein, the subject has a brain malignancy. Brain malignancies that can be treated in accordance with the methods herein include, but are not limited to, glioma, astrocytoma, oligodendroglioma, ependymomas, meningioma, medulloblastoma, ganglioglioma, schwannoma, craniopharyngioma, chordoma, and non-Hodgkin lymphoma.

In some embodiments of the methods disclosed herein, the subject has a head and neck malignancy. Malignancies known collectively as head and neck malignancies usually begin in the squamous cells that line the moist, mucosal surfaces inside the head and neck (e.g., inside the mouth, nose, and throat). Head and neck cancers can also originate in the salivary glands. Exemplary head and neck malignancies include, but are not limited to, squamous cell carcinomas of the oral cavity, pharynx (nasopharynx, oropharynx, hypopharynx), larynx, paranasal sinuses and nasal cavity, and salivary glands.

In some embodiments of the methods disclosed herein, the subject has a blood malignancy. Blood malignancies that can be treated in accordance with the methods herein include, but are not limited to, acute myeloid leukemia, chronic lymphocytic leukemia, chronic myeloid leukemia, acute lymphocytic leukemia, lymphoblastic lymphoma, Burkitt lymphoma, large cell lymphoma, and Hodgkin lymphoma.

In some embodiments of the methods disclosed herein, the subject has a bone malignancy. Bone malignancies that can be treated in accordance with the methods herein include, but are not limited to, osteosarcoma, chondrosarcoma (dedifferentiated chondrosarcoma, clear cell chondrosarcoma, mesenchymal chondrosarcoma), Ewing sarcoma, malignant fibrous histiocytoma, fibrosarcoma, giant cell tumor of bone, chordoma, non-Hodgkin lymphoma, and multiple myeloma.

In some embodiments of the methods disclosed herein, the subject has a thyroid malignancy. Thyroid malignancies that can be treated in accordance with the methods herein include, thyroid malignancies include, but are not limited to, papillary carcinoma, papillary adenocarcinoma, follicular carcinoma, follicular adenocarcinoma, oxyphil cell carcinoma, sporadic medullary thyroid carcinoma, familial medullary thyroid carcinoma, anaplastic thyroid cancer, lymphoma, and sarcoma.

Another aspect of the disclosure herein relates to a method of reducing malignancy-associated fibrosis in a subject. This method involves selecting a subject having a malignancy and administering DMHCA or a derivative thereof to the subject in an amount effective to reduce malignancy-associated fibrosis in the subject. Malignancies suitable for treatment in accordance with this aspect of the disclosure are described supra.

In accordance with this aspect of the disclosure, the selected subject having a malignancy as disclosed herein may additionally exhibit one or more markers of malignancy-associated fibrosis. Malignancy-associated fibrosis is characterized by unchecked pro-fibrotic and pro-inflammatory signaling. Markers of malignancy-associated fibrosis in the tumor microenvironment include, without limitation, the presence of malignancy-associated fibroblasts, dense extracellular collagen deposition, extracellular matrix stiffness, and excess fibrous connective tissue in an organ (see, e.g., Jiang et al., “Tumor-Associated Fibrosis as a Regulator of Tumor Immunity and Response to Immunotherapy,” Cancer Immunol. Immunother. 66(8):1037-1048 (2017), which is hereby incorporated by reference in its entirety). Another marker of malignancy-associated fibrosis is an increase in fibroblast activation protein (FAP), a cell surface serine dipeptidase involved in ECM remodeling, wound healing, inflammation, and tumor growth.

In accordance with the methods described herein, i.e., methods of treating a malignancy in a subject and methods of reducing malignancy-associated fibrosis in a subject, the subject having a malignancy is administered DMHCA or a derivative thereof. DMHCA is a desmosterol analog and a liver X receptor (LXR) agonist that induces cholesterol efflux and inhibits inflammation without inducing SREBF1-dependent lipogenesis and hepatotoxicity (Chaffer & Weinberg, “A Perspective on Cancer Cell Metastasis,” Science 331:1559-1564 (2011), which is hereby incorporated by reference in its entirety). However, as demonstrated herein, it has been found that DMHCA reduces tumor development and tumor multiplicity in animal models of malignancy. This reduction in tumor development and tumor multiplicity is accompanied by a significant enhancement in the anti-tumor immune response and reduction in immune tolerance.

DMHCA, which is also referred to as N,N-dimethyl-3-hydroxy-5-cholenamide and N,N-dimethyl-3-HOChNH₂, has the chemical structure of formula (I).

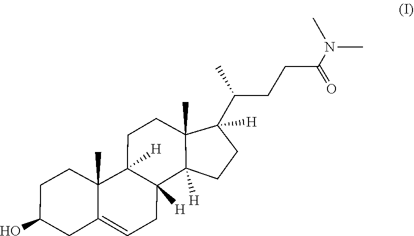

As referred to herein, a “derivative” of DMHCA refers to a salt thereof, a pharmaceutically acceptable salt thereof, an ester thereof, a free acid form thereof, a free base form thereof, a solvate thereof, a deuterated derivative thereof, a hydrate thereof, an N-oxide thereof, a clathrate thereof, a prodrug thereof, a polymorph thereof, a stereoisomer thereof, a geometric isomer thereof, a tautomer thereof, a mixture of tautomers thereof, an enantiomer thereof, a diastereomer thereof, a racemate thereof, a mixture of stereoisomers thereof, an isotope thereof (e.g., tritium, deuterium), or a combination of any of these derivatives.

As used herein, the term “pharmaceutically acceptable salt” refers to a salt prepared from a base or acid which is acceptable for administration to a subject, such as a mammal. The term “pharmaceutically acceptable salts” embraces salts commonly used to form alkali metal salts and to form addition salts of free acids or free bases. The nature of the salt is not critical, provided that it is pharmaceutically-acceptable. Such salts can be derived from pharmaceutically-acceptable inorganic or organic bases and from pharmaceutically-acceptable inorganic or organic acids.

Suitable pharmaceutically acceptable acid addition salts of DMHCA may be prepared from an inorganic acid or an organic acid. All of these salts may be prepared by conventional means from DMHCA by treating, for example, the compound with the appropriate acid or base.

Pharmaceutically acceptable acids include both inorganic acids, for example hydrochloric, hydrobromic, hydroiodic, nitric, carbonic, sulfuric, phosphoric and diphosphoric acid; and organic acids, for example formic, acetic, trifluoroacetic, propionic, succinic, glycolic, embonic (pamoic), methanesulfonic, ethanesulfonic, 2-hydroxyethanesulfonic, pantothenic, benzenesulfonic, toluenesulfonic, sulfanilic, mesylic, cyclohexylaminosulfonic, stearic, algenic, p-hydroxybutyric, malonic, galactic, galacturonic, citric, fumaric, gluconic, glutamic, lactic, maleic, malic, mandelic, mucic, ascorbic, oxalic, pantothenic, succinic, tartaric, benzoic, acetic, xinafoic (1-hydroxy-2-naphthoic acid), napadisilic (1,5-naphthalenedisulfonic acid), and the like.

Salts derived from pharmaceutically-acceptable inorganic bases include aluminum, ammonium, calcium, copper, ferric, ferrous, lithium, magnesium, manganic, manganous, potassium, sodium, zinc, and the like. Salts derived from pharmaceutically-acceptable organic bases include salts of primary, secondary and tertiary amines, including alkyl amines, arylalkyl amines, heterocyclyl amines, cyclic amines, naturally-occurring amines, and the like, such as arginine, betaine, caffeine, choline, chloroprocaine, diethanolamine, N-methylglucamine, N,N′-dibenzylethylenediamine, diethylamine, 2-diethylaminoethanol, 2-dimethylaminoethanol, ethanolamine, ethylenediamine, N-ethylmorpholine, N-ethylpiperidine, glucamine, glucosamine, histidine, hydrabamine, isopropylamine, lysine, methylglucamine, morpholine, piperazine, piperidine, polyamine resins, procaine, purines, theobromine, triethylamine, trimethylamine, tripropylamine, tromethamine, and the like.

Other preferred salts according to embodiments herein are quaternary ammonium compounds wherein an equivalent of an anion (X−) is associated with the positive charge of the N atom. X− may be an anion of various mineral acids such as, for example, chloride, bromide, iodide, sulphate, nitrate, phosphate, or an anion of an organic acid such as, for example, acetate, maleate, fumarate, citrate, oxalate, succinate, tartrate, malate, mandelate, trifluoroacetate, methanesulphonate and p-toluenesulphonate. X− is preferably an anion selected from chloride, bromide, iodide, sulphate, nitrate, acetate, maleate, oxalate, succinate or trifluoroacetate.

As disclosed herein, a derivative of DMHCA also includes an N-oxide thereof. An N-oxide is formed from the tertiary basic amines or imines present in the molecule, using a convenient oxidizing agent.

A derivative of DMHCA also includes unsolvated and solvated forms. The term solvate is used herein to describe a molecular complex comprising DMHCA and an amount of one or more pharmaceutically acceptable solvent molecules. The term hydrate is employed when said solvent is water. Examples of solvate forms include, but are not limited to, DMHCA in association with water, acetone, dichloromethane, 2-propanol, ethanol, methanol, dimethylsulfoxide (DMSO), ethyl acetate, acetic acid, ethanolamine, or mixtures thereof. It is specifically contemplated that in embodiments herein one solvent molecule can be associated with one molecule of DMHCA, such as a hydrate.

Furthermore, it is specifically contemplated that in embodiments herein, more than one solvent molecule may be associated with one molecule of DMHCA, such as a dihydrate. Additionally, it is specifically contemplated that in embodiments herein less than one solvent molecule may be associated with one molecule of DMHCA, such as a hemihydrate. Furthermore, solvates of embodiments herein are contemplated as solvates of DMHCA that retain the biological effectiveness of the non-solvate form of the compounds.

A derivative of DMHCA also includes isotopically-labeled compounds, wherein one or more atoms is replaced by an atom having the same atomic number, but an atomic mass or mass number different from the atomic mass or mass number usually found in nature. Examples of isotopes suitable for inclusion in the compounds of embodiments herein include isotopes of hydrogen, such as 2H and 3H; isotopes of carbon, such as 11C, 13C and 14C; isotopes of nitrogen, such as 13N and 15N; and isotopes of oxygen, such as 150, 170 and 180. Certain isotopically-labeled DMHCA, for example, those incorporating a radioactive isotope, are useful in drug and/or substrate tissue distribution studies. The radioactive isotopes tritium (3H) and carbon-14 (14C) are particularly useful for this purpose in view of their ease of incorporation and ready means of detection. Substitution with heavier isotopes such as deuterium (2H) may afford certain therapeutic advantages resulting from greater metabolic stability, for example, increased in vivo half-life or reduced dosage requirements, and hence may be preferred in some circumstances. Substitution with positron emitting isotopes, such as 11C, 150 and 13N, can be useful in Positron Emission Topography (PET) studies for examining substrate receptor occupancy.

Isotopically-labeled DMHCA can generally be prepared by conventional techniques known to those skilled in the art or by processes analogous to those described herein, using an appropriate isotopically-labeled reagent in place of the non-labeled reagent otherwise employed.

Preferred isotopically-labeled compounds include deuterated derivatives of the compounds of embodiments herein. As used herein, the term deuterated derivative embraces DMHCA where in a particular position at least one hydrogen atom is replaced by deuterium. Deuterium (D or 2H) is a stable isotope of hydrogen which is present at a natural abundance of 0.015 molar %. Hydrogen deuterium exchange (deuterium incorporation) is a chemical reaction in which a covalently bonded hydrogen atom is replaced by a deuterium atom. Said exchange (incorporation) reaction can be total or partial.

A derivative of DMHCA also includes a prodrug of the compound. A prodrug of DMHCA is a derivative having little or no pharmacological activity itself when administered into the body, but which is converted into DMHCA having the desired activity, for example, by hydrolytic cleavage. Prodrugs in accordance with embodiments herein can, for example, be produced by replacing appropriate functionalities present in DMHCA with certain moieties known to those skilled in the art as “pro-moieties” as described in VIVEKKUMAR REDASANI & SANJAY BARI, PRODRUG DESIGN: PERSPECTIVES, APPROACHES AND APPLICATIONS IN MEDICINAL CHEMISTRY (1st ed. 2015), which is hereby incorporated by reference in its entirety.

In some embodiments of the methods of treating a malignancy and methods of reducing malignancy-associated fibrosis as described herein, DMHCA or a derivative thereof is administered to a subject in an amount effective to reduce or prevent growth of the malignancy. In accordance with these embodiments, “growth of the malignancy” encompasses any aspect of the growth, proliferation, and progression of the malignancy and/or malignant tumor cells, including, e.g., cell division (i.e., mitosis), cell growth (e.g., increase in cell size), an increase in genetic material (e.g., prior to cell division), and metastasis. A reduction, inhibition, prevention, and/or suppression of the malignancy and/or malignant tumor cell growth includes, but is not limited to, a reduction, inhibition, prevention, and/or suppression of the malignancy and/or malignant tumor cell growth as compared to the growth of an untreated or mock treated malignancy and/or malignant tumor cells; a reduction, inhibition, prevention, and/or suppression of malignant cell proliferation; a reduction, inhibition, prevention, and/or suppression of malignant metastasis; a reduction, inhibition, prevention, and/or suppression of malignancy and/or malignant tumor cell size. In some embodiments, DMHCA or a derivative thereof is administered to a subject in an amount effective to induce malignant tumor cell senescence and/or malignant tumor cell death.

In some embodiments of the methods of treating a malignancy and methods of reducing malignancy-associated fibrosis as described herein, the administering is carried out in an amount effective to reduce malignancy multiplicity. As used herein, the term “malignancy multiplicity” refers to the number of malignant tumors in a particular organ or inclusively at any site. In some embodiments, DMHCA or a derivative thereof is administered in an amount effective to reduce malignancy multiplicity by at least 2-fold (e.g., by at least 2-fold, by at least 3-fold, by at least 4-fold, by at least 5-fold, by at least 6-fold, by at least 7-fold, by at least 8-fold, by at least 9-fold, by at least 10-fold, by at least 15-fold, by at least 20-fold, >20-fold; e.g., in a range having a lower limit selected from 2-fold, 3-fold, 4-fold, 5-fold, 6-fold, 7-fold, 8-fold, 9-fold, 10-fold, and 15-fold, and an upper limit selected from 3-fold, 4-fold, 5-fold, 6-fold, 7-fold, 8-fold, 9-fold, 10-fold, 15-fold, and 20-fold, in any combination thereof).

As demonstrated herein, it has been found that DMHCA significantly enhances the anti-tumor immune response in animal models of malignancy and fibrosis-associated malignancy. The immune response typically mounts a response against malignant cells as a first line of defense against the malignant growth. An effective antitumor immune response requires processing of tumor-associated antigens by dendritic cells (DC), presentation of antigens to antigen-specific T cells, activation and proliferation of those T cells, and maintenance of the T-cell response long enough for the T cells to effectively eliminate the malignancy (Makkouk et al., “Cancer Immunotherapy and Breaking Immune Tolerance: New Approaches to an Old Challenge,” Cancer Res. 75(1):5-10 (2015), which is hereby incorporated by reference in its entirety). However, malignant cells have developed multiple mechanisms, including alteration of the antigen presentation machinery or secretion of immunosuppressive factors, that induce apoptosis of lymphocytes or activate negative regulatory pathways to induce tolerance and limit the effectiveness of the immune response (Makkouk et al., “Cancer Immunotherapy and Breaking Immune Tolerance: New Approaches to an Old Challenge,” Cancer Res. 75(1):5-10 (2015), which is hereby incorporated by reference in its entirety). Changes in the tumor microenvironment can also contribute to the suppression of adaptive immunity or immune tolerance (Ma et al., “Gene Expression Profiling of the Tumor Microenvironment During Breast Cancer Progression,” Breast Cancer Res. 11:R7 (2009); Shimizu et al., “Immune Suppression and Reversal of the Suppressive Tumor Microenvironment,” Int. Immunol. 30(10):445-454 (2018), each of which is hereby incorporated by reference in its entirety).

Thus in accordance with the methods of treating a malignancy or reducing malignancy-associated fibrosis in a subject as described herein, the malignancy may be a malignancy that exhibits immune tolerance. In some embodiments, the malignancy treated in accordance with the methods described herein is resistant to treatment with an immune checkpoint inhibitor. As used herein, the term “immune checkpoint inhibitor” refers to a drug that blocks certain proteins made by immune system cells (e.g., T cells) and some malignant tumor cells. These proteins help keep immune responses in check and can keep T cells from killing the malignancies and/or malignant tumor cells, i.e., they promote immune tolerance. When these proteins are blocked, the “brakes” on the immune system are released and T cells are able to kill the malignancies and/or malignant tumor cells better. Examples of immune checkpoint inhibitors include programmed cell death protein 1 (PD-1) inhibitors, programmed death-ligand 1 (PD-L1) inhibitors, cytotoxic T-lymphocyte-associated protein 4 (CTLA-4) inhibitors, B7-1 inhibitors, and B7-2 inhibitors. Thus, in some embodiments, the malignancy to be treated in accordance with the methods described herein is resistant to treatment with one or more immune checkpoint inhibitors.

In accordance with these embodiments, the amount of DMHCA administered to the subject to treat the malignancy and/or reduce malignancy-associated fibrosis is an amount effective to enhance the subject's anti-malignancy immune response. In some embodiments, DMHCA is administered in an amount effective to increase the number and/or activity of CD4⁺ and CD8⁺ effector T cells, monocytes, and/or macrophages in the tumor microenvironment. In some embodiments, DMHCA is administered in an amount effective to reduce the number and/or function of regulatory T cells (i.e., Treg cells), granulocytic myeloid derived suppressor cells (G-MDSCs), and monocytic myeloid-derived suppressor cells (M-MDSCs) in the tumor microenvironment.

In some embodiments the methods of treating a malignancy or reducing malignancy-associated fibrosis in a subject as described herein, involve administering DMHCA in an amount effective to prevent metastasis of the malignancy. As used herein, the term “metastasis” refers to the spread of malignant cells from the place where they first formed to another part of the body. In metastasis, malignant cells break away from the original (primary) tumor, travel through the blood or lymph system, and form a new tumor in other organs or tissues of the body. The new, metastatic tumor is the same type of malignancy as the primary tumor. For example, if a breast malignancy spreads to the lung, the malignant cells in the lung are breast malignancy cells, not lung malignancy cells.

In accordance with the methods of treating a malignancy or reducing malignancy-associated fibrosis in a subject as described herein, the DMHCA or a derivative thereof is administered at a dose ranging from about 0.1 mg/kg to about 1000 mg/kg body weight (e.g., ˜0.1 mg/kg, ˜0.5 mg/kg, ˜1 mg/kg, ˜2.5 mg/kg, ˜5 mg/kg, ˜7.5 mg/kg, ˜10 mg/kg, ˜20 mg/kg, ˜30 mg/kg, ˜40 mg/kg, ˜50 mg/kg, ˜60 mg/kg, ˜70 mg/kg, ˜80 mg/kg, ˜90 mg/kg, ˜100 mg/kg, ˜125 mg/kg, ˜150 mg/kg, ˜175 mg/kg, ˜200 mg/kg, ˜225 mg/kg, ˜250 mg/kg, ˜275 mg/kg, ˜300 mg/kg, ˜325 mg/kg, ˜350 mg/kg, ˜375 mg/kg, ˜400 mg/kg, ˜425 mg/kg, ˜450 mg/kg, ˜475 mg/kg, ˜500 mg/kg, ˜550 mg/kg, ˜600 mg/kg, ˜650 mg/kg, ˜700 mg/kg, ˜750 mg/kg, ˜800 mg/kg, ˜850 mg/kg, ˜900 mg/kg, ˜950 mg/kg, ˜1000 mg/kg; e.g., in a range having a lower limit selected from ˜0.1 mg/kg, ˜0.5 mg/kg, ˜1 mg/kg, ˜2.5 mg/kg, ˜5 mg/kg, ˜7.5 mg/kg, ˜10 mg/kg, ˜20 mg/kg, ˜30 mg/kg, ˜40 mg/kg, ˜50 mg/kg, ˜60 mg/kg, ˜70 mg/kg, ˜80 mg/kg, ˜90 mg/kg, ˜100 mg/kg, ˜125 mg/kg, ˜150 mg/kg, ˜175 mg/kg, ˜200 mg/kg, ˜225 mg/kg, ˜250 mg/kg, ˜275 mg/kg, ˜300 mg/kg, ˜325 mg/kg, ˜350 mg/kg, ˜375 mg/kg, ˜400 mg/kg, ˜425 mg/kg, ˜450 mg/kg, ˜475 mg/kg, ˜500 mg/kg, ˜550 mg/kg, ˜600 mg/kg, ˜650 mg/kg, ˜700 mg/kg, ˜750 mg/kg, ˜800 mg/kg, ˜850 mg/kg, ˜900 mg/kg, and ˜950 mg/kg, and an upper limit selected from ˜0.5 mg/kg, ˜1 mg/kg, ˜2.5 mg/kg, ˜5 mg/kg, ˜7.5 mg/kg, ˜10 mg/kg, ˜20 mg/kg, ˜30 mg/kg, ˜40 mg/kg, ˜50 mg/kg, ˜60 mg/kg, ˜70 mg/kg, ˜80 mg/kg, ˜90 mg/kg, ˜100 mg/kg, ˜125 mg/kg, ˜150 mg/kg, ˜175 mg/kg, ˜200 mg/kg, ˜225 mg/kg, ˜250 mg/kg, ˜275 mg/kg, ˜300 mg/kg, ˜325 mg/kg, ˜350 mg/kg, ˜375 mg/kg, ˜400 mg/kg, ˜425 mg/kg, ˜450 mg/kg, ˜475 mg/kg, ˜500 mg/kg, ˜550 mg/kg, ˜600 mg/kg, ˜650 mg/kg, ˜700 mg/kg, ˜750 mg/kg, ˜800 mg/kg, ˜850 mg/kg, ˜900 mg/kg, ˜950 mg/kg, and ˜1000 mg/kg, in any combination thereof). The exact dosage to be administered will depend on the characteristics of the subject being treated, e.g., the type and stage of the malignancy, the level of immune tolerance exhibited by the malignancy, the age, weight, and health of the subject, types of concurrent treatment, if any, and frequency of treatments, and can be readily determined by one of skill in the art (e.g., by the clinician).

In some embodiments of the methods described herein, the administering is repeated periodically. For example, the DMHCA or a derivative thereof may be administered at a set interval, e.g., daily, every other day, weekly, biweekly, or monthly. Alternatively, the DMHCA or derivative thereof is administered at an irregular interval, for example on an as-needed basis based on symptoms, patient health, and the like. For example, an effective amount may be administered once a day (q.d.) for one day, at least 2 days, at least 3 days, at least 4 days, at least 5 days, at least 6 days, at least 7 days, at least 10 days, at least 15 days, at least 30 days, at least 1 month, at least 2 months, at least 3 months, at least 4 months, at least 5 months, at least 6 months, at least 7 months, at least 8 months, at least 9 months, at least 10 months, at least 11 months, at least 12 months, or >12 months (e.g., for a duration having a lower limit selected from one day, 2 days, 3 days, 4 days, 5 days, 6 days, 7 days, 10 days, 15 days, 30 days, 1 month, 2 months, 3 months, 4 months, 5 months, 6 months, 7 months, 8 months, 9 months, 10 months, 11 months, and 12 months, and an upper limit selected from 2 days, 3 days, 4 days, 5 days, 6 days, 7 days, 10 days, 15 days, 30 days, 1 month, 2 months, 3 months, 4 months, 5 months, 6 months, 7 months, 8 months, 9 months, 10 months, 11 months, 12 months, and greater than 12 months, in any combination thereof).

As demonstrated herein, DMHCA has been shown to enhance the anti-tumor immune response and reduce immune tolerance in malignancies. Because immune tolerance is generally present in all solid tumors, it is expected that combining any anti-cancer therapeutic agent with DMHCA can improve the treatment outcome as compared to administering either agent alone. Thus, in some embodiments of the method of treating a malignancy and the method of reducing malignancy-associated fibrosis described herein, the method further involves administering at least one anti-cancer therapeutic agent to said subject in combination with said DMHCA or a derivative thereof. Suitable anti-cancer therapeutic agents in accordance with these methods include immunotherapeutic agents, chemotherapeutic agents, radiotherapies, vaccines, anti-inflammatory agents, gene targeting agents, and the like.

In some embodiments of the method of treating a malignancy and the method of reducing malignancy-associated fibrosis described herein, the method further involves administering at least one immunotherapeutic agent to said subject in combination with said DMHCA or a derivative thereof. As used herein, immunotherapeutic agents are substances that stimulate the immune system to help fight a malignancy. In some embodiments, the immunotherapeutic agent is an immune checkpoint inhibitor. Suitable immune checkpoint inhibitors include, for example, anti-PD-L1 immunotherapeutic agents, anti-CTLA-4 (cytotoxic T-lymphocyte-associated protein 4) immunotherapeutic agents, anti-LAG-3 (lymphocyte activation gene 3) immunotherapeutic agents, anti-TIGIT (T cell immunoreceptor with Ig and ITIM domains) immunotherapeutic agents, and the like.

In some embodiments, the immunotherapeutic agent is an anti-PD-1 immunotherapeutic agent or an anti-PD-L1 immunotherapeutic agent. Suitable anti-PD-1 immunotherapeutic agents include, but are not limited to, nivolumab (Opdivo®) and pembrolizumab (Keytruda®). Suitable anti-PD-L1 immunotherapeutic agents include, but are not limited to, atezolizumab (MPDL3280A) and durvalumab (MEDI4736).

In some embodiments, the immunotherapeutic agent is an anti-CTLA-4 immunotherapeutic agent. In the immune recognition process, two signals are required for T lymphocyte expansion and differentiation: the T-cell receptor (TCR) binding to the HLA molecule-peptide complex and an antigen-independent costimulatory signal provided by the B7 (CD80 and Cd86)/CD28 interaction. CTLA-4 is a homologous molecule of CD28 that is a competitive antagonist for B7. CTLA-4 has a greater affinity and avidity for B7 than does CD28, and its translocation to the cell surface after T-cell activation results in B7 sequestration and transduction of a negative signal, responsible for T-cell inactivation (Pérez-Garcia et al., “CTLA-4 Polymorphisms and Clinical Outcome After Allogeneic Stem Cell Transplantation from HLA-Identical Sibling Donors,” Blood 110(1):461-467 (2007), which is hereby incorporated by reference in its entirety). Thus, inhibition of CTLA-4 enhances T-cell activation, amplifies T-cell proliferation, and promotes the generation of memory T cells.

Suitable CTLA-4 immunotherapeutic agents include, but are not limited to, Ipilimumab (Yervoy®), Tremelimumab, and AGEN1884.

Suitable anti-LAG-3 immunotherapeutic agents include, but are not limited to, relatlimab (BMS-986016).

Suitable anti-TIGIT immunotherapeutic agents include, but are not limited to, the anti-TIGIT monoclonal antibody BMS-986207.

In all aspects of the present disclosure that involve administering combination(s) of therapeutic agents, e.g., administering DMHCA or a derivative thereof in combination with an immunotherapeutic agent as described supra, DMHCA or a derivative thereof may be administered before, during, or after the administration of any, some, or all of the other therapeutic agents described herein. In some embodiments, administering the at least one immunotherapeutic agent occurs simultaneously with administering DMHCA or a derivative thereof. In other embodiments, administering the at least one immunotherapeutic agent occurs separately from administering DMHCA or a derivative thereof.

In some embodiments, the therapeutic agents described herein are administered on the same day, about 24 hours apart, about 23 hours apart, about 22 hours apart, about 21 hours apart, about 20 hours apart, about 19 hours apart, about 18 hours apart, about 17 hours apart, about 16 hours apart, about 15 hours apart, about 14 hours apart, about 13 hours apart, about 12 hours apart, about 11 hours apart, about 10 hours apart, about 9 hours apart, about 8 hours apart, about 7 hours apart, about 6 hours apart, about 5 hours apart, about 4 hours apart, about 3 hours apart, about 2 hours apart, about 1 hour apart, about 55 minutes apart, about 50 minutes apart, about 45 minutes apart, about 40 minutes apart, about 35 minutes apart, about 30 minutes apart, about 25 minutes apart, about 20 minutes apart, about 15 minutes apart, about 10 minutes apart, or about 5 minutes apart. In some embodiments, the therapeutic agents described herein are administered about 1 day apart, about 2 days apart, about 3 days apart, about 4 days apart, about 5 days apart, about 6 days apart, or about 1 week apart.

In some embodiments of the methods described herein, the method further involves administering a chemotherapeutic agent to the subject in combination with DMHCA or a derivative thereof. Suitable chemotherapeutic agents to be administering in combination with DMHCA or a derivative thereof include, without limitation, paclitaxel, docetaxel, albumin-bound paclitaxel, epirubicin, doxorubicin, pegylated liposomal doxorubicin, 5-fluorouracil, cyclophosphamide, cisplatin, carboplatin, vinorelbine, capecitabine, gemcitabine, ixabepilone, eribulin, cyclophosphamide, chorambucil and other alkylating agents, vinblastine, vincristine, irinotecan, ispinesib, filanesib and other motor protein inhibitors, barasertib, danusertib and other aurora kinase A and B inhibitors, polo kinase inhibitors, mipomersen, nusinersen and other antisense oligonucleotides, tamoxifen, raloxifene and other hormone receptor antagonists, letrozole, anastrozole and other aromatase inhibitors, imatinib, dasatinib, ponatinib, bosutinib, axitinib, tozasertib, ava pritinib and other tyrosine kinase inhibitors, erlotinib, gefitinib, osimertinib and other EGF receptor kinase inhibitors and monocloncal antibodies, ibrutinib, acalabrutinib and other Bruton kinase inhibitors, venetoclax and other BCL2/BH3 inhibitors, idealasib and other PI3K inhibitors, BRAF inhibitors, MEK inhibitors, VEGF receptor kinase inhibitors and monoclonal antibodies, angiogenesis receptor and angiogenesis targeted inhibitors, perifosine and other AKT inhibitors, MET receptor inhibitors, HER2 receptor monoclonal antibodies, IGF receptor monoclonal antibodies, bevacizumab and other VEGF monoclonal antibodies, ipilimumab, pembrolizumab, nivolumab, atezolizumab, avelumab and other immune checkpoint monoclonal antibodies, CAR-T therapies, 4-1BB, CD40 and other immune cell-targeted antibodies, chemokine receptor antagonists, glucocorticoid receptor agonists, cytokines, NSAIDS, PPAR agonists, and any combination thereof.

The methods of treating a malignancy and reducing malignancy associated fibrosis as described herein can be carried out on any subject having a malignancy. In one embodiment, the subject is a mammal. Suitable mammals include, without limitation, primates (e.g., humans, monkeys), equines (e.g., horses), bovines (e.g., cattle), porcines (e.g., pigs), ovines (e.g., sheep), caprines (e.g., goats), camelids (e.g., llamas, alpacas, camels), rodents (e.g., mice, rats, guinea pigs, hamsters), canines (e.g., dogs), felines (e.g., cats), and leporids (e.g., rabbits). In some embodiments, the mammalian subject is a human subject. In some embodiments, the mammal subject is an agricultural animal, a domesticated animal, a zoo animal, or a laboratory animal.

As will be apparent to the skilled artisan, the therapeutic agents may be administered using any suitable method. By way of example, suitable modes of administration include, without limitation, orally, topically, transdermally, parenterally, intradermally, intrapulmonary, intramuscularly, intraperitoneally, intravenously, subcutaneously, or by intranasal instillation, by intracavitary or intravesical instillation, intraocularly, intraarterialy, intralesionally, or by application to mucous membranes. In some embodiments of the methods described herein, the DMHCA is administered orally.

Suitable modes of local administration of the therapeutic agents and/or combinations disclosed herein include, without limitation, catheterization, implantation, direct injection, dermal/transdermal application, or portal vein administration to relevant tissues, or by any other local administration technique, method or procedure generally known in the art. The mode of affecting delivery of agent will vary depending on the type of therapeutic agent and the malignancy to be treated.

In certain embodiments, the therapeutic agents described herein may be administered as part of a single formulation or separate formulation. In either embodiment, the present disclosure also relates to kits in which DMHCA, an immunotherapeutic agent, and a chemotherapeutic agent are contained together, for example as a copackaging arrangement, with instructions to administer them to the selected subject described herein.

Another aspect of the disclosure described herein relates to a pharmaceutical combination. This composition comprises DMHCA or a derivative thereof, and one or more immune checkpoint inhibitors.

In some embodiments, the one or more immune checkpoint inhibitors of the pharmaceutical combination described herein, include a PD-1 inhibitor, a PD-L1 inhibitor, or a CTLA-4 inhibitor. Suitable PD-1 inhibitors, PD-L1 inhibitors, and CTLA-4 inhibitors are described above.

In some embodiments, the pharmaceutical combination described herein comprises dimethyl-3-beta-hydroxy-cholenamide (DMHCA) or a derivative thereof, and one or more immune checkpoint inhibitors formulated for simultaneous administration of. In some embodiments, the pharmaceutical combination described herein comprises dimethyl-3-beta-hydroxy-cholenamide (DMHCA) or a derivative thereof, and one or more immune checkpoint inhibitors formulated for separate administration.

Preferences and options for a given aspect, feature, embodiment, or parameter of the technology described herein should, unless the context indicates otherwise, be regarded as having been disclosed in combination with any and all preferences and options for all other aspects, features, embodiments, and parameters of the technology.

The present technology may be further illustrated by reference to the following examples.

### EXAMPLES

The following examples are provided to illustrate embodiments of the present technology but are by no means intended to limit its scope.

### Materials and Methods for Examples 1-5

### Animals

The derivation and characteristics of MMTV-PPARd mice was reported in Yuan et al., “PPARdelta Induces Estrogen Receptor-Positive Mammary Neoplasia Through an Inflammatory and Metabolic Phenotype Linked to mTOR Activation,” Cancer Res. 73(14):4349-4361 (2013), which is hereby incorporated by reference in its entirety. MMTV-NeuT mice (Muller et al., “Single-Step Induction of Mammary Adenocarcinoma in Transgenic Mice Bearing the Activated c-neu Oncogene,” Cell 54(1):105-115 (1988), which is hereby incorporated by reference in its entirety) were obtained from Jackson Labs (FVB-Tg(MMTV-Erbb2)NK1Mul/J); they exhibit mammary tumorigenesis by 6-7 months (Guy et al., “Induction of Metastatic Mammary Tumors by Expression of Polyoma Middle T Oncogene: A Transgenic Mouse Model for Metastatic Disease,” Mol. Cell Biol. 12(3):954-961 (1992), which is hereby incorporated by reference in its entirety). FAT-ATTAC mice on a C57BL/6 background (Pajvani et al., “Fat Apoptosis Through Targeted Activation of Caspase 8: A New Mouse Model of Inducible and Reversible Lipoatrophy,” Nat. Med. 11(7):797-803 (2005); Landskroner-Eiger et al., “Morphogenesis of the Developing Mammary Gland. Stage-Dependent Impact of Adipocytes,” Dev. Biol. 344(2):968-978 (2010), each of which is hereby incorporated by reference in its entirety) were crossed into the FVB strain for several generations. Derivation of NeuT/ATTAC mice was reported in Yuan et al., “MMTV-NeuT/ATTAC Mice: A New Model for Studying the Stromal Tumor Microenvironment,” Oncotarget. 9(8):8042-8053 (2018), which is hereby incorporated by reference in its entirety. All animals were fed LabDiet 5303 and monitored for the NeuT, PPARd, and/or FKBPv-caspase 8 transgenes by genotyping.

### Treatments

Fibrosis was induced in mice at 6 w.o.a. by weekly i.p. injections of 0.4 mg/kg AP dissolved in vehicle (4% ethanol, 10% PEG-400, and 1.75% Tween-20 in water). Mice were maintained on LabDiet 5053 chow supplemented with 0.05% DMHCA (100 mg/kg) beginning at 8 w.o.a. Control mice received the standard diet.

### Histopathology and Immunohistochemistry

Mammary tissue was excised and FFPE sections were prepared (Yuan et al., “MMTV-NeuT/ATTAC Mice: A New Model for Studying the Stromal Tumor Microenvironment,” Oncotarget. 9(8):8042-53 (2018); Yuan et al., “PPARdelta Induces Estrogen Receptor-Positive Mammary Neoplasia Through an Inflammatory and Metabolic Phenotype linked to mTOR Activation,” Cancer Res. 73(14):4349-4361 (2013); Yin et al., “Peroxisome Proliferator-Activated Receptor Delta and Gamma Agonists Differentially Alter Tumor Differentiation and Progression During Mammary Carcinogenesis,” Cancer Res. 65:3950-3957 (2005); Yin et al., “Inhibition of Peroxisome Proliferator-Activated Receptor Gamma Increases Estrogen Receptor-Dependent Tumor Specification,” Cancer Res. 69(2):687-694 (2009), each of which is hereby incorporated by reference in its entirety). Primary antibodies: PD-L1 (1:200, 17952-1-AP, Proteintech), SMA (1:100, sc130617, Santa Cruz), Ki67 (1:50, CRM325, Biocare), CD31 (1:100, ab56299, Abcam), CXCL1 (1:120, sc-1374, Santa Cruz), F4/80 (1:30, 14-4801-85, eBioscience), Foxp3 (1:50, 14-5773-82, e Bioscience), CD8a (1:40, 14-0808-82, eBioscience). Collagen was stained with Picrosirius Red stain (Yuan et al., “MMTV-NeuT/ATTAC Mice: A New Model for Studying the Stromal Tumor Microenvironment,” Oncotarget. 9(8):8042-53 (2018), which is hereby incorporated by reference in its entirety).

### Statistical Analysis

Statistical significance was evaluated using the Mantel-Cox log-rank test for survival analysis and Prism GraphPad software or the two-tailed Student's t test to evaluate differences between means at a significance of P<0.05.

### Example 1—NeuT/ATTAC and PPARd/ATTAC Genetically Engineered Models (GEM) of Fibrosis

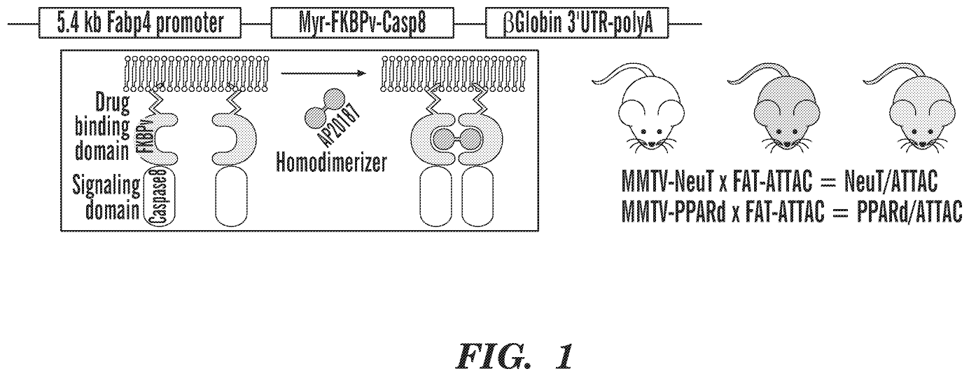

To address the role of fibrosis in cancer progression, two conditional genetically engineered mouse models, i.e., PPARd/ATTAC and NeuT/ATTAC mice, were utilized in the studies described herein (see FIG. 1) (Yuan et al., “MMTV-NeuT/ATTAC Mice: A New Model for Studying the Stromal Tumor Microenvironment,” Oncotarget. 9(8):8042-8053 (2018); Guy et al., “Activated Neu Induces Rapid Tumor Progression,” J. Biol. Chem. 271(13):7673-7678 (1996); Muller et al., “Single-Step Induction of Mammary Adenocarcinoma in Transgenic Mice Bearing the Activated C-Neu Oncogene,” Cell 54(1):105-115 (1988); Yuan et al., “Ablation of the Mammary Fat Microenvironment Accelerates Tumorigenesis in MMTV-PPARd Mice,” Keystone Symposium on Obesity and Adipose Tissue Biology, Banff, Alberta, Canada, February 15-19, 2016, each of which is hereby incorporated by reference in its entirety). These models allow for the delineation of temporal, histologic, metabolic, and molecular changes occurring in fibrosis, and also serve as models for therapeutic intervention.

Previous attempts to modify the mammary TME in C3(1)-Tag transgenic mice crossed into the “fatless” dominant-negative A-ZIP/F-1 background (Kim et al., “Mechanism of Insulin Resistance in A-ZIP/F-1 Fatless Mice,” J. Biol. Chem. 275:8456-8460 (2000), which is hereby incorporated by reference in its entirety) resulted in increased tumorigenesis, but also produced a severe diabetic and systemic inflammatory condition due to the ablation of all body fat, making it difficult to determine which condition contributed to cancer progression (Nunez et al., “Accelerated Tumor Formation in a Fatless Mouse with Type 2 Diabetes and Inflammation,” Cancer Res. 66:5469-5476 (2006), which is hereby incorporated by reference in its entirety).

To address this concern, the FAT-ATTAC mouse was developed, in which white fat could be conditionally ablated by forced dimerization and activation of the FKBPv-caspase fusion protein by the dimerizer AP21087 (AP) (Pajvani et al., “Fat Apoptosis Through Targeted Activation of Caspase 8: A New Mouse Model of Inducible and Reversible Lipoatrophy,” Nat. Med. 11(7):797-803 (2005); Landskroner-Eiger et al., “Morphogenesis of the Developing Mammary Gland. Stage-Dependent Impact of Adipocytes,” Dev. Biol. 344(2):968-978 (2010), each of which is hereby incorporated by reference in its entirety) (see FIG. 1). The affinity of AP for the mutated FKBPv is 1,000-fold greater than for endogenous FKBP (Clackson et al., “Redesigning an FKBP-Ligand Interface to Generate Chemical Dimerizers with Novel Specificity,” Proc. Natl. Acad. Sci. USA 95:10437-10442 (1998), which is hereby incorporated by reference in its entirety), and thus highly selective for the transgene. Loss of varying amounts of adipose tissue following caspase activation results in infiltration and proliferation of stromal fibroblasts and myoepithelial cells (Pajvani et al., “Fat Apoptosis Through Targeted Activation of Caspase 8: A New Mouse Model of Inducible and Reversible Lipoatrophy,” Nat. Med. 11(7):797-803 (2005), which is hereby incorporated by reference in its entirety), and in female mice, affects only the mammary fat pad without producing diabetes (Landskroner-Eiger et al., “Morphogenesis of the Developing Mammary Gland. Stage-Dependent Impact of Adipocytes,” Dev. Biol. 344(2):968-978 (2010), which is hereby incorporated by reference in its entirety).

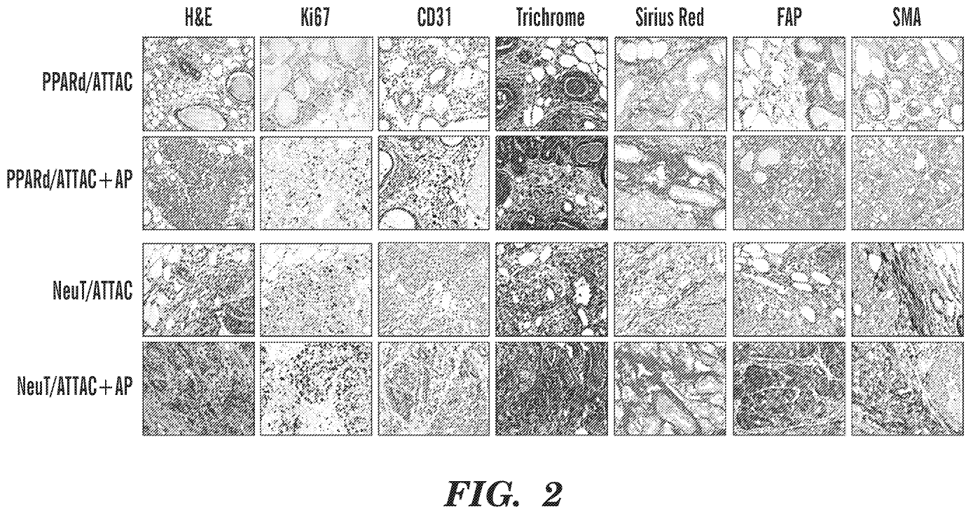

FAT-ATTAC mice (ATTAC) were crossed with MMTV-NeuT mice, which express a constitutively active rat ErbB2 gene containing the V664E point mutation that results in ductal mammary tumors resembling the HER2⁺/ER7 subtype (Guy et al., “Activated neu Induces Rapid Tumor Progression,” J. Biol. Chem. 271(13):7673-7678 (1996); Muller et al., “Single-Step Induction of Mammary Adenocarcinoma in Transgenic Mice Bearing the Activated c-neu Oncogene,” Cell 54(1):105-115 (1998), each of which is hereby incorporated by reference in its entirety), to produce NeuT/ATTAC mice (FIG. 1). FAT-ATTAC mice were crossed with MMTV-PPARd mice to produce PPARd/ATTAC mice (FIG. 1). In NeuT/ATTAC mice, induction of fibrosis by treatment with AP accelerated tumor development and increased tumor multiplicity (Lin et al., “Targeting Liver X receptors in Cancer Therapeutics,” Nat. Rev. Cancer 15:216-224 (2015), which is hereby incorporated by reference in its entirety), and similar fibrotic changes occurred in PPARd/ATTAC mice (Yuan et al., “Ablation of the Mammary Fat Microenvironment Accelerates Tumorigenesis in MMTV-PPARd Mice,” Keystone Symposium on Obesity and Adipose Tissue Biology, Banff, Alberta, Canada, February 15-19, 2016, which is hereby incorporated by reference in its entirety) (FIG. 2). Both GEM models exhibited an immunotolerant phenotype associated with an acute phase inflammatory response characteristic of invasive cancers (Ghavami et al., “S100A8/A9: A Janus-Faced Molecule in Cancer Therapy and Tumorgenesis,” Eur. J. Pharmacol. 625:73-83 (2009); Malle et al., “Serum Amyloid A: An Acute-Phase Protein Involved in Tumour Pathogenesis,” Cell. Mol. Life Sci. 66:9-26 (2009), each of which is hereby incorporated by reference in its entirety), including breast cancer (Nasser et al., “RAGE Mediates S100A7-Induced Breast Cancer Growth and Metastasis by Modulating the Tumor Microenvironment,” Cancer Res. 75:974-985 (2015), which is hereby incorporated by reference in its entirety).

MMTV-PPARd mice are unique in that unlike most GEM models (Herschkowitz et al., “Identification of Conserved Gene Expression Features Between Murine Mammary Carcinoma Models and Human Breast Tumors,” Genome Biol. 8:R76 (2007), which is hereby incorporated by reference in its entirety), they develop adenocarcinomas resembling the luminal B subtype (Yuan et al., “PPARdelta Induces Estrogen Receptor-Positive Mammary Neoplasia Through an Inflammatory and Metabolic Phenotype linked to mTOR Activation,” Cancer Res. 73(14):4349-4361 (2013), which is hereby incorporated by reference in its entirety), which are particularly difficult to treat (Ellis & Perou, “The Genomic Landscape of Breast Cancer as a Therapeutic Roadmap,” Cancer Discov. 3:27-34 (2013), which is hereby incorporated by reference in its entirety). PPARd is a ligand-dependent nuclear receptor that like LXR also plays a multi-faceted role in metabolism, inflammation, and neoplasia (Barish et al., “PPAR delta: A Dagger in the Heart of the Metabolic Syndrome,” J. Clin. Invest. 116:590-597 (2006); Wagner & Wagner, “Peroxisome Proliferator-Activated Receptor beta/delta (PPARbeta/delta) Acts as Regulator of Metabolism Linked to Multiple Cellular Functions,” Pharmacol. Ther. 125:423-435 (2010); Michalik et al., “Peroxisome-Proliferator-Activated Receptors and Cancers: Complex Stories,” Nat. Rev. Cancer 4:61-70 (2004); Glazer et al., “PPARgamma and PPARdelta as Modulators of Neoplasia and Cell Fate,” PPAR Res. 2008:247379 (2008); Peters et al., “The Role of Peroxisome Proliferator-Activated Receptors in Carcinogenesis and Chemoprevention,” Nat. Rev. Cancer 12:181-95 (2012), each of which is hereby incorporated by reference in its entirety). PPARd agonists facilitate mammary tumorigenesis (Yuan et al., “PPARdelta Induces Estrogen Receptor-Positive Mammary Neoplasia Through an Inflammatory and Metabolic Phenotype Linked to mTOR Activation,” Cancer Res. 73(14):4349-4361 (2013); Pollock et al., “PPARdelta Activation Acts Cooperatively with 3-Phosphoinositide-Dependent Protein Kinase-1 to Enhance Mammary Tumorigenesis,” PloS One 6:e16215 (2011); Yin et al., “Peroxisome Proliferator-Activated Receptor Delta and Gamma Agonists Differentially Alter Tumor Differentiation and Progression During Mammary Carcinogenesis,” Cancer Res. 65:3950-3957 (2005), each of which is hereby incorporated by reference in its entirety) and gastric neoplasia (Pollock et al., “Induction of Metastatic Gastric Cancer by Peroxisome Proliferator-Activated Receptordelta Activation,” PPAR Res. 2010:571783 (2010), which is hereby incorporated by reference in its entirety), in part by upregulating phosphoinositide-dependent protein kinase 1 (PDPK1/PDK1), a major regulatory kinase involved in AKT, protein kinase C, and mTOR signaling (Pearce et al., “The Nuts and Bolts of AGC Protein Kinases,” Nat. Rev. Mol. Cell Biol. 11:9-22 (2010), which is hereby incorporated by reference in its entirety). Metabolomic analysis of mammary tumors from MMTV-PPARd mice demonstrated that tumorigenesis was mTOR-dependent via increased lysophosphatidic acid biosynthesis (Foster, “Phosphatidic Acid Signaling to mTOR: Signals for the Survival of Human Cancer Cells,” Biochem. Biophys. Aca. 1791:949-955 (2009), which is hereby incorporated by reference in its entirety), the same pathway responsible for breast cancer progression (Jonkers & Moolenaar, “Mammary Tumorigenesis Through LPA Receptor Signaling,” Cancer Cell 15:457-459 (2009); Panupinthu et al., “Lysophosphatidic Acid Production and Action: Critical New Players in Breast Cancer Initiation and Progression,” Br. J. Cancer 102:941-946 (2010), which is hereby incorporated by reference in its entirety) and metabolic reprogramming of cancer-associated fibroblasts (Valencia et al, “Metabolic Reprogramming of Stromal Fibroblasts Through p62-mTORC1 Signaling Promotes Inflammation and Tumorigenesis,” Cancer Cell 26:121-35 (2014), which is hereby incorporated by reference in its entirety). PPARd activation in the mammary gland increased arachidonic acid and long chain fatty acid synthesis (Yuan et al., “PPARdelta Induces Estrogen Receptor-Positive Mammary Neoplasia Through an Inflammatory and Metabolic Phenotype Linked to mTOR Activation,” Cancer Res. 73(14):4349-61 (2013); Pollock et al., “PPARdelta Activation Acts Cooperatively with 3-Phosphoinositide-Dependent Protein Kinase-1 to Enhance Mammary Tumorigenesis,” PloS One 6:e16215 (2011), each of which is hereby incorporated by reference in its entirety), which promoted their interaction with fatty acid-binding proteins (FABP) (Storch & Thumser, “Tissue-Specific Functions in the Fatty Acid-Binding Protein Family,” J. Biol. Chem. 285:32679-83 (2010), which is hereby incorporated by reference in its entirety) and its ability to potentiate EGFR- and ErbB2-dependent proliferation (Kannan-Thulasiraman et al., “Fatty Acid-Binding Protein 5 and PPARbeta/delta Are Critical Mediators of Epidermal Growth Factor Receptor-Induced Carcinoma Cell Growth,” J. Biol. Chem. 285:19106-19115 (2010); Levi et al., “Genetic Ablation of the Fatty Acid-Binding Protein FABP5 Suppresses HER2-Induced Mammary Tumorigenesis,” Cancer Res. 73(15):4770-4780 (2013), each of which is hereby incorporated by reference in its entirety). From a clinical perspective, the oncogenic role of PPARd is consistent with its increased mRNA and protein expression in invasive breast cancer (Glazer et al., “PPARgamma and PPARdelta as Modulators of Neoplasia and Cell Fate,” PPAR Res. 2008:247379 (2008); Abdollahi et al., “Transcriptional Network Governing the Angiogenic Switch in Human Pancreatic Cancer,” Proc. Natl. Acad. Sci. USA 104:12890-12895 (2007), each of which is hereby incorporated by reference in its entirety) and as a predictor of poor survival (Kittler et al., “A Comprehensive Nuclear Receptor Network for Breast Cancer Cells,” Cell Reports 3:538-551 (2013), which is hereby incorporated by reference in its entirety). Although, PPARd has also been implicated in promoting other epithelial cancers such as colon cancer (Wang et al., “Peroxisome Proliferator-Activated Receptor Delta Promotes Colonic Inflammation and Tumor Growth,” Proc. Natl. Acad. Sci. USA 111:7084-7089 (2014), which is hereby incorporated by reference in its entirety), its role in this context remains controversial due to differences in the various knockout mouse models used (Glazer et al., “PPARgamma and PPARdelta as Modulators of Neoplasia and Cell Fate,” PPAR Res. 2008:247379 (2008); Peters et al., “The Role of Peroxisome Proliferator-Activated Receptors in Carcinogenesis and Chemoprevention,” Nat. Rev. Cancer 12:181-195 (2012); Zuo et al., “Targeted Genetic Disruption of Peroxisome Proliferator-Activated Receptor-Delta and Colonic Tumorigenesis,” J. Natl. Cancer Inst. 101:762-767 (2009); Park & Kwak, “The Role of Peroxisome Proliferator-Activated Receptors in Colorectal Cancer,” PPAR Res. 2012:876418 (2012), each of which is hereby incorporated by reference in its entirety).

These models, which afford conditional mammary tumorigenesis, also allow for the induction of varying degrees of fibrosis specifically in the mammary gland (Yuan et al., “MMTV-NeuT/ATTAC Mice: A New Model for Studying the Stromal Tumor Microenvironment,” Oncotarget. 9(8):8042-8053 (2018), which is hereby incorporated by reference in its entirety). IHC analysis of tumor tissue in both models revealed an increase in neoplasia (H&E), proliferation (Ki67), and angiogenesis (CD31), as well as collagen deposition (Trichrome stain & Sirius Red stain), FAP, and SMA, all hallmarks of fibrosis (FIG. 2; NeuT/ATTAC data reported in Yuan et al., “MMTV-NeuT/ATTAC Mice: A New Model for Studying the Stromal Tumor Microenvironment,” Oncotarget. 9(8):8042-8053 (2018), which is hereby incorporated by reference in its entirety).

Fibrosis in NeuT/ATTAC mice was accompanied by increased CXCL1, CCL7, CCL2 associated with monocyte and neutrophil mobilization and MDSC activation, as well as PD-L1 expression (Lin et al., “Targeting Liver X receptors in Cancer Therapeutics,” Nat. Rev. Cancer 15: 216-224 (2015), which is hereby incorporated by reference in its entirety), which emphasizes the critical role of fibrosis in activating inflammatory and immunomodulatory factors and tumor progression.

### Example 2—HER2⁺ Breast Cancer Biopsies Exhibit Collagen Deposition and FAP Expression

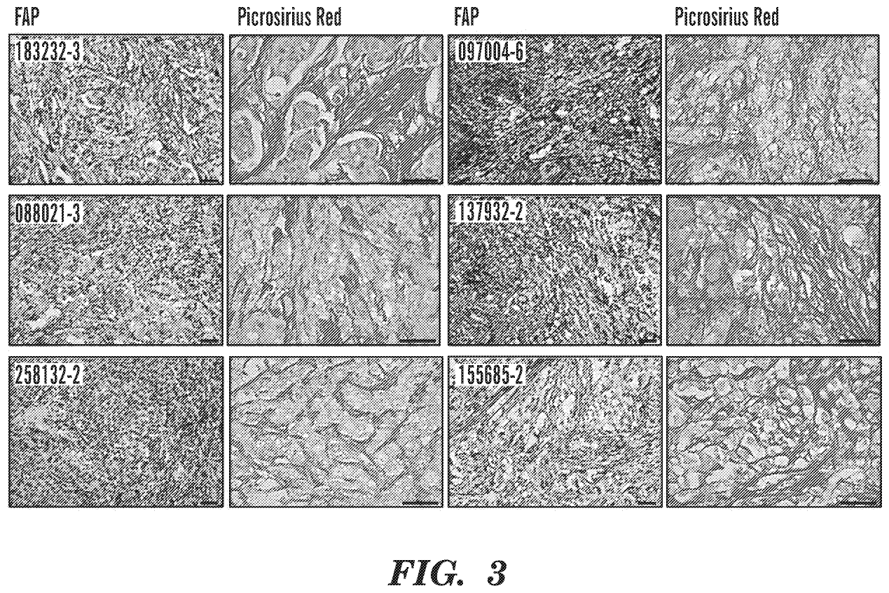

To emphasize the relevance of fibrosis in NeuT/ATTAC mice to HER2⁺ breast cancer, 12 biopsies from HER2⁺ breast cancer patients were analyzed for collagen expression by Picrosirius Red staining and for FAP by IHC (FIG. 3 (six representative images are shown)). All tumor biopsies exhibited varying degrees of collagen deposition and FAP expression, indicating that fibrosis in the TME is a common condition in HER2⁺ breast cancer.

### Example 3—Collagen Gene Expression in NeuT/ATTAC and PPARd/ATTAC AP-Induced Fibrotic Tumor Tissue

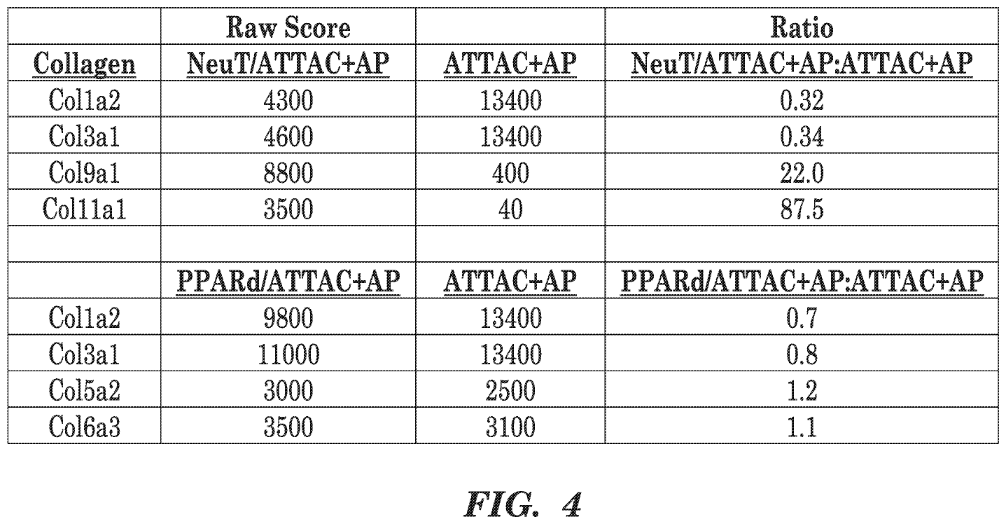

Next, collagen gene expression associated with fibrotic tumor tissue from NeuT/ATTAC and PPARd/ATTAC mice following AP treatment was compared to collagen expression in the fibrotic mammary gland of non-tumorigenic FAT-ATTAC (“ATTAC”) mice treated with AP (FIG. 4). These data indicate that Col1a2, Col 3a1, Col6a3, Col11a1, and Col5a2 are similarly increased in the NeuT/ATTAC and PPARd/ATTAC models and breast cancer (Naba et al., “The Extracellular Matrix: Tools and Insights for the ‘Omics’ Era,” Matrix Biol. 49:10-24 (2016), which is hereby incorporated by reference in its entirety), suggesting important differences between fibrotic normal mammary gland and tumor.

### Example 4—DMHCA Treatment Studies in NeuT/ATTAC and PPARd/ATTAC GEM

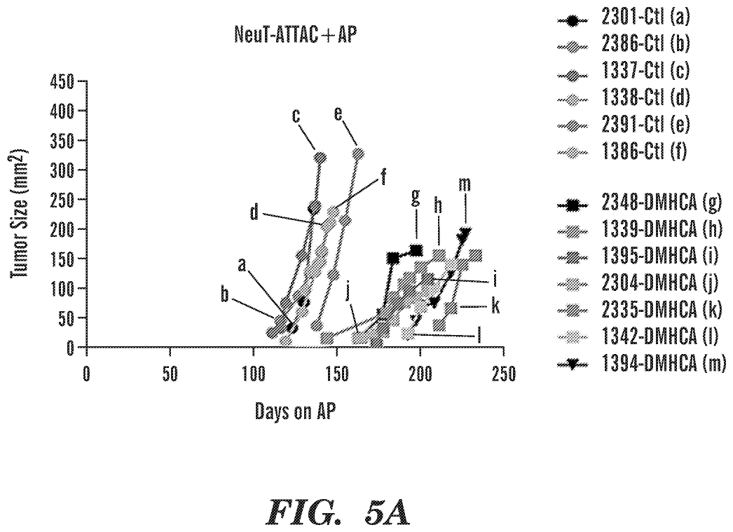

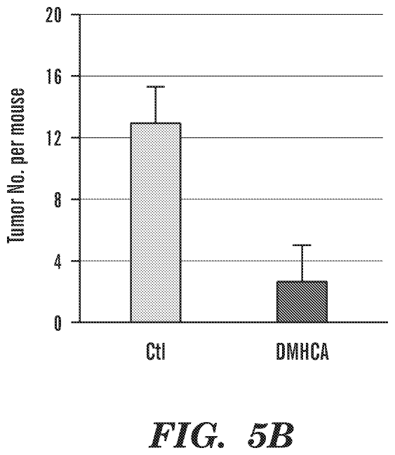

A study was designed to evaluate whether DMHCA treatment could abrogate tumor development in NeuT/ATTAC mice treated with AP. Remarkably, mice maintained on a diet supplemented with 0.05% DMHCA (˜100 mg/kg/day, and nontoxic) showed a reduction in tumor development (FIG. 5A) and a 10-fold decrease in tumor multiplicity (FIG. 5B).

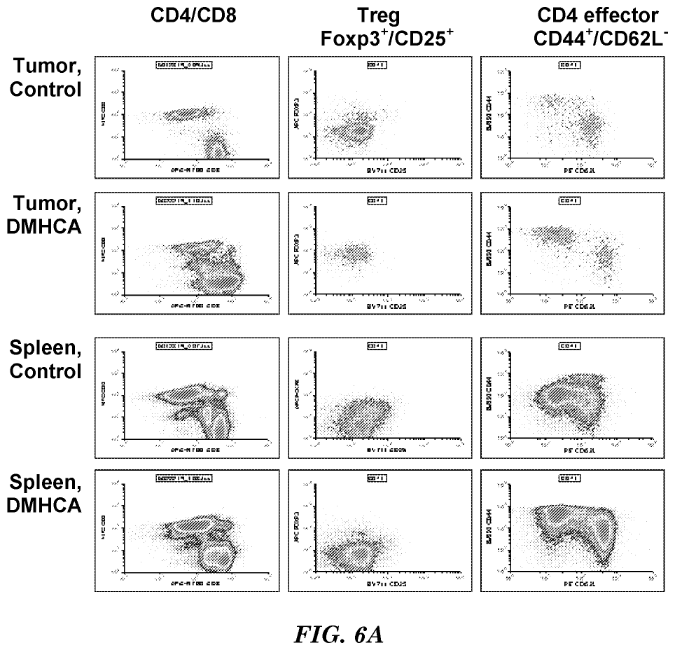

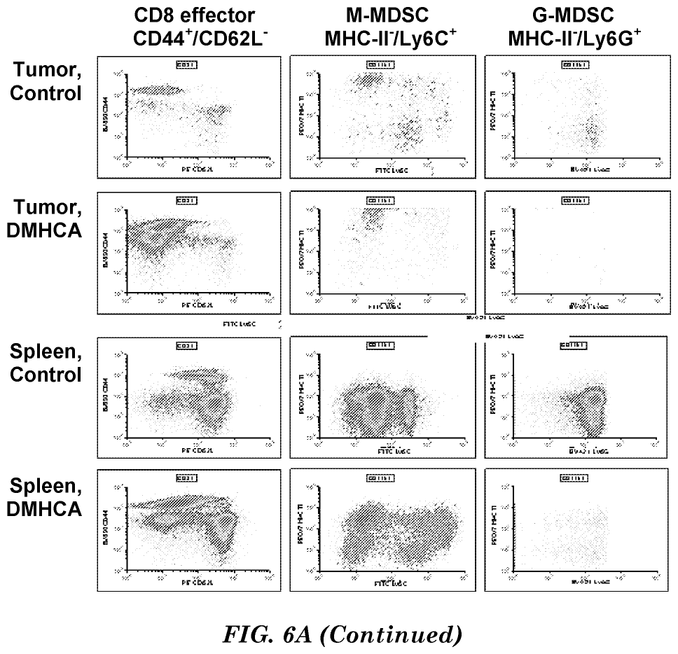

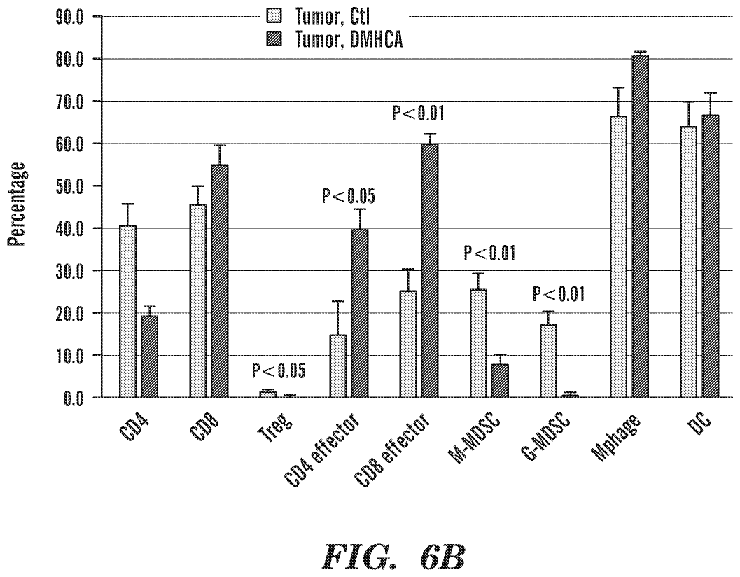

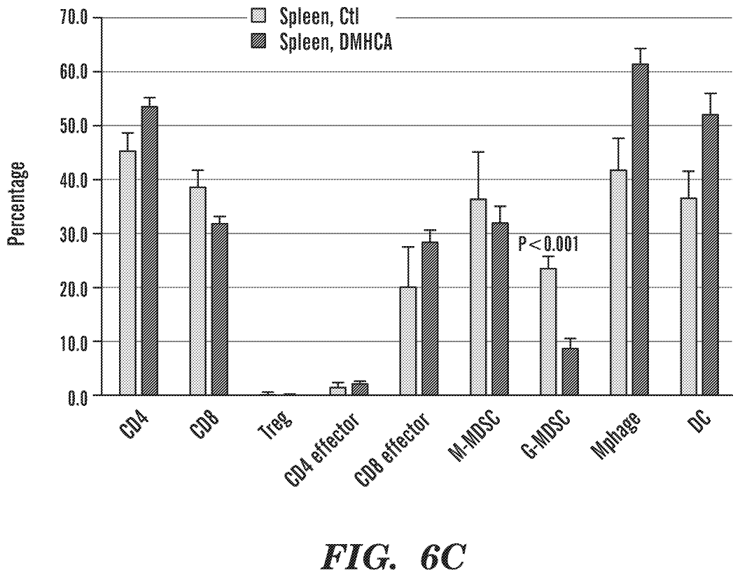

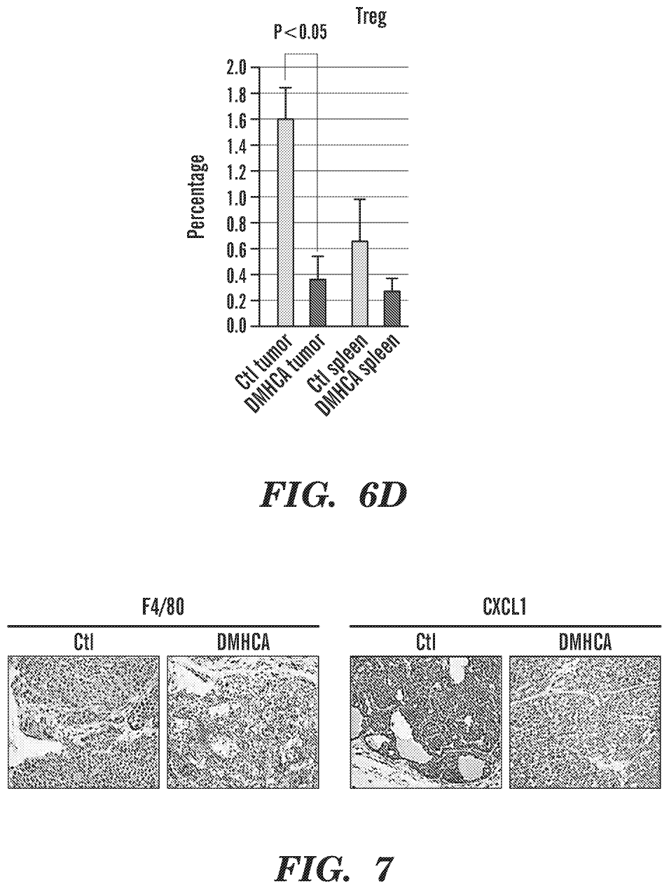

To assess whether DMHCA treatment could reduce immune tolerance in NeuT/ATTAC mice, a model that is highly resistant to PD-1 immunotherapy, spleen and tumor immune infiltrates were analyzed by FACS (FIGS. 6A-6C). FACS analysis of immune cell subsets in tumor infiltrates revealed that DMHCA increased CD4⁺ and CD8⁺ effector T cells and reduced Treg, M-MDSC, and G-MDSC in tumor infiltrates, and reduced G-MDSC in spleen (FIGS. 6A-6D). Additionally, reduction in tumor growth was associated with increased macrophage infiltration and reduced CXCL1 expression as assessed by IHC (FIG. 7).

### Example 5—FLIM and SHG Analysis of Tumor Section from DMHCA-Treated Mice

Fibrotic tissue consists mainly of fibronectins and collagens that accumulate in many pathological conditions (Wynn & Ramalingam, “Mechanisms of Fibrosis: Therapeutic Translation for Fibrotic Disease,” Nat. Med. 18(7):1028-1040 (2012); Rosenbloom et al., “Human Fibrotic Diseases: Current Challenges in Fibrosis Research,” Methods Mol. Biol. 1627:1-23 (2017); Ho et al., “Fibrosis-A Lethal Component of Systemic Sclerosis,” Nat. Rev. Rheumatol. 10(7):390-402 (2014), each of which is hereby incorporated by reference in its entirety). Among these proteins, collagens are the most abundant proteins in the human body, and consist of fibrillar (types I, IL, III, V and XI) and non-fibrillar forms (the remaining subtypes), which are responsible for tensile strength and tissue flexibility, respectively (Ricard-Blum, “The Collagen Family,” Cold Spring Harb. Perspect. Biol. 3(1):a004978 (2011), which is here by incorporated by reference in its entirety). The most abundant fibrillary collagen is type I, which is non-centrosymmetric and often co-distributed with type III. Collagens are detected by Masson's Trichrome staining and Picrosirius Red staining, IHC, UPLC/MS and second harmonic generation (SHG) microscopy.

SHG imaging using a multiphoton microscope equipped with a Deep Imaging Via Emission Recovery (DIVER) detector (Crosignani et al., “Deep Tissue Fluorescence Imaging and in Vivo Biological Applications,” J. Biomed. Opt. 17(11):116023 (2012), which is hereby incorporated by reference in its entirety) can distinguish collagen accumulation in the early stages of fibrosis unlike standard histological analysis (Dvornikov & Gratton, “Imaging in Turbid Media: A Transmission Detector Gives 2-3 Order of Magnitude Enhanced Sensitivity Compared to Epi-Detection Schemes,” Biomed. Opt. Express 7:3747-755 (2016); Ranjit et al., “Label-Free Fluorescence Lifetime and Second Harmonic Generation Imaging Microscopy Improves Quantification of Experimental Renal Fibrosis,” Kidney Int. 90:1123-1128 (2016), each of which is hereby incorporated by reference in its entirety). DIVER, by virtue of detection in the direction of light propagation is especially suitable for SHG detection and a combination of SHG and FLIM enables spatial mapping of collagen I and III, which is important for fibrosis. Additionally, this type of imaging is applicable to unstained tissues, which eliminates the uncertainty from issues associated with labeling efficiency. Moreover, the same tissue can be used for other purposes after imaging is complete. In live cells, phasor-FLIM allows for measurement and quantification of inflammation (Alfonso-Garcia et al., “Label-Free Identification of Macrophage Phenotype by Fluorescence Lifetime Imaging Microscopy,” J. Biomed. Opt. 21:46005 (2016), which is hereby incorporated by reference in its entirety), oxidative stress (Datta et al., “Fluorescence Lifetime Imaging of Endogenous Biomarker of Oxidative Stress,” Sci. Rep. 5:9848 (2015), which is hereby incorporated by reference in its entirety), metabolism (Ranjit et al., “Determination of the Metabolic Index Using the Fluorescence Lifetime of Free and Bound Nicotinamide Adenine Dinucleotide Using the Phasor Approach,” J. Biophotonics 12:e201900156 (2019), which is hereby incorporated by reference in its entirety), and cholesterol (Malacrida et al., “A Multidimensional Phasor Approach Reveals LAURDAN Photophysics in NIH-3T3 Cell Membranes,” Sci. Rep. 7:9215 (2017), which is hereby incorporated by reference in its entirety) accumulation, which enables the study of physiological changes with high spatial resolution. Metabolic FLIM imaging enables examination of the subpopulation of cells where Warburg effect is more prominent and deciphering whether that subpopulation decreases with treatment.

Spatial mapping by SHG microscopy was used to distinguish depth differentiation between normal, dysplastic, and malignant tissue in a DMBA mammary tumor model (Guo et al., “Subsurface Tumor Progression Investigated by Noninvasive Optical Second Harmonic Tomography,” Proc. Nal. Acad. Sci. USA 96:10854-10856 (1999), which is hereby incorporated by reference in its entirety), and to acquire 3D information in mammary tumors from MMTV-PyMT and MMTV-Wntl transgenic mice, that allowed depiction of tumor cells oriented along radially aligned collagen fibers during invasion (Provenzano et al., “Collagen Reorganization at the Tumor-Stromal Interface Facilitates Local Invasion,” BMC Med. 4:38 (2006), which is hereby incorporated by reference in its entirety). Similar information has been obtained to distinguish basal cell carcinoma and ovarian cancer from normal tissue (Lin et al., “Discrimination of Basal Cell Carcinoma from Normal Dermal Stroma by Quantitative Multiphoton Imaging,” Opt. Lett. 31:2756-2758 (2006); Nadiarnykh et al., “Alterations of the Extracellular Matrix in Ovarian Cancer Studied by Second Harmonic Generation Imaging Microscopy,” BMC Cancer 10:94 (2010), each of which is hereby incorporated by reference in its entirety), that further emphasizes the usefulness of SHG microscopy for imaging the fibrotic TME. The presence of collagens and NADH/FAD in a variety of tissues can be detected using fluorescence lifetime imaging microscopy (FLIM) based on their endogenous autofluorescence characteristics (Ranjit et al., “Label-Free Fluorescence Lifetime and Second Harmonic Generation Imaging Microscopy Improves Quantification of Experimental Renal Fibrosis,” Kidney Int. 90:1123-1128 (2016); Stringari et al., “Phasor Fluorescence Lifetime Microscopy of Free and Protein-Bound NADH Reveals Neural Stem Cell Differentiation Potential,” PloS One 7:e48014 (2012); Stringari et al., “Metabolic Trajectory of Cellular Differentiation in Small Intestine by Phasor Fluorescence Lifetime Microscopy of NADH,” Sci. Rep. 2:568 (2012); Wright et al., “NADH Distribution in Live Progenitor Stem Cells by Phasor-Fluorescence Lifetime Image Microscopy,” Biophys. J. 103:L7-L9 (2012); Ranjit et al., “Measuring the Effect of a Western Diet on Liver Tissue Architecture by FLIM Autofluorescence and Harmonic Generation Microscopy,” Biomed. Opt. Express 8:3143-3154 (2017); Ranjit et al., “Characterizing Fibrosis in UUO Mice Model Using Multiparametric Analysis of Phasor Distribution from FLIM Images,” Biomed. Opt. Express 7:3519-3530 (2016); Ranjit et al., “Imaging Fibrosis and Separating Collagens Using Second Harmonic Generation and Phasor Approach to Fluorescence Lifetime Imaging,” Sci. Rep. 5:13378 (2015), each of which is hereby incorporated by reference in its entirety). Fluorescence lifetimes of free (0.4 ns) and protein-bound NADH (3.4 ns) can be easily distinguished, and their relative molar fractions vary as a function of cellular metabolism, where a higher fraction of free NADH is indicative of glycolytic metabolism, whereas a higher fraction of protein-bound NADH is indicative of oxidative metabolism. FLIM has been used to measure glycolysis in several breast cancer cell lines (Bird et al., “Metabolic Mapping of MCF10A Human Breast Cells via Multiphoton Fluorescence Lifetime Imaging of the Coenzyme NADH,” Cancer Res. 65:8766-8773 (2005); Cannon et al., “Autofluorescence Imaging Captures Heterogeneous Drug Response Differences Between 2D and 3D Breast Cancer Cultures,” Biomed. Opt. Express 8:1911-1925 (2017), each of which is hereby incorporated by reference in its entirety) and to distinguish metabolic changes in HER2⁺ breast cancer xenografts in response to trastuzumab (Walsh et al., “Optical Metabolic Imaging Identifies Glycolytic Levels, Subtypes, and Early-Treatment Response in Breast Cancer,” Cancer Res. 73:6164-6174 (2013), which is hereby incorporated by reference in its entirety).

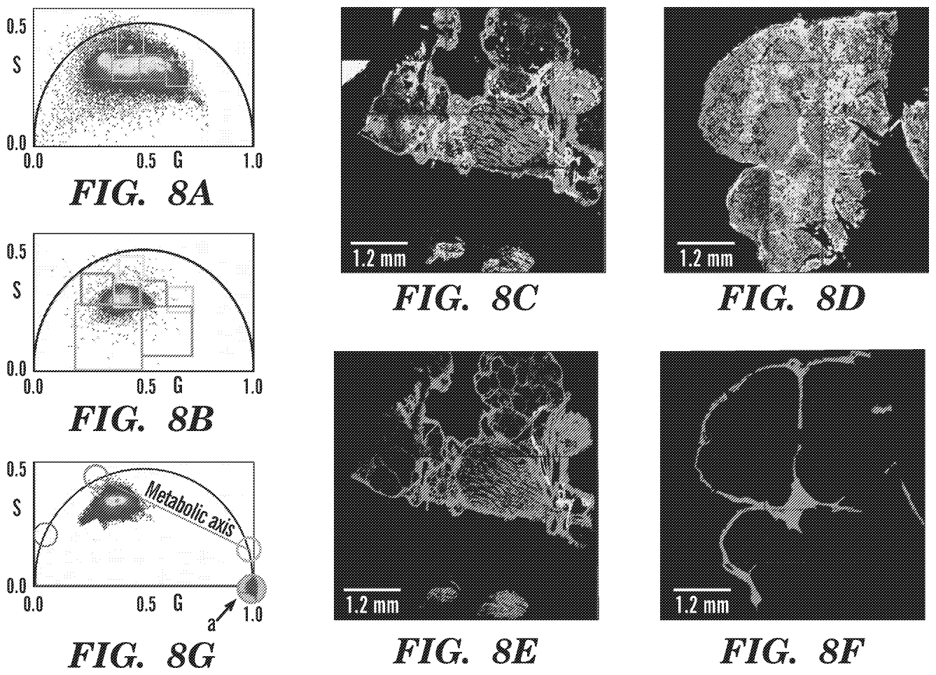

SHG imaging was combined with FLIM and the phasor approach to characterize fibrosis generated by collagens types I/III in NeuT/ATTAC mice treated with DMHCA (FIGS. 8A-8G). Combining SHG and FLIM provides a label-free, nondestructive method for 3D imaging of fibrosis in living tissues and tissue sections.

FLIM detected collagen I & collagen III in the control tumor (FIG. 8C), but little collagen in the DMHCA-treated tumor (FIG. 8D). The phasor plot showed a greater abundance of bound NADH in the control tumor (FIG. 8A) in comparison to higher levels of free NADH after DMHCA treatment (FIG. 8B).

SHG microscopy demonstrated that collagen I in the control tumor was markedly reduced after DMHCA treatment (FIGS. 8E-8G).

### Discussion of Examples 1-5

Administration of the LXR agonist DMHCA to NeuT/ATTAC mice beginning at two months of age reduced tumorigenesis, tumor multiplicity, and fibrosis as shown by reduced collagen, fibroblast activation protein, and smooth muscle actin expression. SHG microscopy confirmed the reduction of collagen deposition that was accompanied by an increase in free NADH as determined by FLIM. Reduction of fibrosis resulted in a marked decrease of MDSC infiltration and an increase in CD8⁺ effector T cells and PD1⁺/Foxp3⁺ CD4⁺ T cells.

The results presented herein establish a connection between the reversal of fibrosis and reduced tumorigenesis by DMHCA, which is accompanied by a reduction in immune tolerance.

Although preferred embodiments have been depicted and described in detail herein, it will be apparent to those skilled in the relevant art that various modifications, additions, substitutions, and the like can be made without departing from the spirit of the invention and these are therefore considered to be within the scope of the invention as defined in the claims which follow.

What is claimed is:

1. A method of treating a malignancy in a subject, said method comprising:
selecting a subject having a malignancy, and
administering dimethyl-3-beta-hydroxy-cholenamide (DMHCA) or a derivative thereof to the subject in an amount effective to treat the malignancy.

2. The method of claim 1, wherein the malignancy exhibits immune tolerance and the amount is effective to enhance the subject's anti-malignancy immune response, thereby treating the malignancy.

3. The method of claim 1 or claim 2, wherein said administering is carried out in an amount effective to reduce or prevent growth of the malignancy.

4. The method of any one of claims 1-3, wherein said administering is carried out in an amount effective to reduce malignancy multiplicity.

5. A method of reducing malignancy-associated fibrosis in a subject, said method comprising:
selecting a subject having a malignancy; and
administering dimethyl-3-beta-hydroxy-cholenamide (DMHCA) or a derivative thereof to the subject in an amount effective to reduce malignancy-associated fibrosis in the subject.

6. The method of claim 5, wherein the selected subject has one or more markers of malignancy-associated fibrosis.

7. The method of any one of claims 1-6, wherein the malignancy is a breast malignancy, pancreatic malignancy, lung malignancy, liver malignancy, gastrointestinal malignancy, esophageal malignancy, colorectal malignancy, renal malignancy, bladder malignancy, prostate malignancy, cervical malignancy, testicular malignancy, skin malignancy, brain malignancy, head and neck malignancy, blood cell malignancy, bone malignancy, thyroid malignancy, stomach malignancy, gallbladder malignancy, or ovarian malignancy.

8. The method of claim 7, wherein the malignancy is a breast malignancy.

9. The method of claim 8, wherein the breast malignancy is an HER2⁺/ER⁺ malignancy.

10. The method of any one of claims 1-9, wherein the malignancy is resistant to treatment with an immune checkpoint inhibitor.

11. The method of claim 10, wherein the immune checkpoint inhibitor is a Programmed Cell Death Protein 1 (PD-1) inhibitor or a Programmed Death-Ligand 1 (PD-L1) inhibitor.

12. The method of any one of claims 1-11, wherein said administering is carried out in an amount effective to prevent metastasis of the malignancy.

13. The method of any one of claims 1-12, wherein the DMHCA or derivative thereof is administered at a dose ranging from 0.1 mg/kg to 1000 mg/kg.

14. The method of any one of claims 1-13, wherein said administering is repeated periodically.

15. The method of any one of claims 1-14, wherein said method further comprises:
administering at least one anti-cancer therapeutic agent to the subject in combination with the DMHCA or derivative thereof.

16. The method of claim 15, wherein the anti-cancer therapeutic agent is an immunotherapeutic agent, a chemotherapeutic agent, a radiotherapy, a vaccine, an anti-inflammatory agent, or a gene targeting agent.

17. The method of claim 16, wherein the anti-cancer therapeutic agent is an immunotherapeutic agent.

18. The method of claim 17, wherein the immunotherapeutic agent is an anti-PD-1 immunotherapeutic agent or an anti-PD-L1 immunotherapeutic agent.

19. The method of claim 17, wherein the immunotherapeutic agent is an anti-Cytotoxic T-Lymphocyte-Associated Protein 4 (CTLA-4) immunotherapeutic agent.

20. The method of claim 16, wherein the anti-cancer therapeutic agent is a chemotherapeutic agent.

21. The method of claim 20, wherein the chemotherapeutic agent is selected from the group consisting of paclitaxel, docetaxel, albumin-bound paclitaxel, epirubicin, doxorubicin, pegylated liposomal doxorubicin, 5-fluorouracil, cyclophosphamide, cisplatin, carboplatin, vinorelbine, capecitabine, gemcitabine, ixabepilone, eribulin, cyclophosphamide, chorambucil and other alkylating agents, vinblastine, vincristine, irinotecan, ispinesib, filanesib and other motor protein inhibitors, barasertib, danusertib and other aurora kinase A and B inhibitors, polo kinase inhibitors, mipomersen, nusinersen and other antisense oligonucleotides, tamoxifen, raloxifene and other hormone receptor antagonists, letrozole, anastrozole and other aromatase inhibitors, imatinib, dasatinib, ponatinib, bosutinib, axitinib, tozasertib, ava pritinib and other tyrosine kinase inhibitors, erlotinib, gefitinib, osimertinib and other EGF receptor kinase inhibitors and monocloncal antibodies, ibrutinib, acalabrutinib and other Bruton kinase inhibitors, venetoclax and other BCL2/BH3 inhibitors, idealasib and other PI3K inhibitors, BRAF inhibitors, MEK inhibitors, VEGF receptor kinase inhibitors and monoclonal antibodies, angiogenesis receptor and angiogenesis targeted inhibitors, perifosine and other AKT inhibitors, MET receptor inhibitors, HER2 receptor monoclonal antibodies, IGF receptor monoclonal antibodies, bevacizumab and other VEGF monoclonal antibodies, ipilimumab, pembrolizumab, nivolumab, atezolizumab, avelumab and other immune checkpoint monoclonal antibodies, CAR-T therapies, 4-1BB, CD40 and other immune cell-targeted antibodies, chemokine receptor antagonists, glucocorticoid receptor agonists, cytokines, NSAIDS, and PPAR agonists.

22. The method of any one of claims 15-21, wherein said administering the at least one anti-cancer therapeutic agent occurs simultaneously with said administering the DMHCA or derivative thereof.

23. The method of any one of claims 15-21, wherein said administering the at least one anti-cancer therapeutic agent occurs separately from said administering the DMHCA or derivative thereof.

24. The method of any one of claims 1-23, wherein the subject is a mammal.

25. The method of claim 24, wherein the mammal is selected from the group consisting of primates (e.g., humans, monkeys), equines (e.g., horses), bovines (e.g., cattle), porcines (e.g., pigs), ovines (e.g., sheep), caprines (e.g., goats), camelids (e.g., llamas, alpacas, camels), rodents (e.g., mice, rats, guinea pigs, hamsters), canines (e.g., dogs), felines (e.g., cats), and leporids (e.g., rabbits),

26. The method of claim 24 or claim 25, wherein the mammal is an agricultural animal, a domesticated animal, a zoo animal, or a laboratory animal.

27. The method of claim 24 or claim 25, wherein the subject is a human.

28. The method of any one of claims 1-27, wherein the DMHCA or derivative thereof is administered orally.

29. A pharmaceutical combination comprising:
(i) dimethyl-3-beta-hydroxy-cholenamide (DMHCA) or derivative thereof, and
(ii) one or more immune checkpoint inhibitors.

30. The pharmaceutical combination of claim 29, wherein the one or more immune checkpoint inhibitors include a PD-1 inhibitor, a PD-L1 inhibitor, or a CTLA-4 inhibitor.

31. The pharmaceutical combination of claim 29 or claim 30, wherein the combination is formulated for simultaneous administration of (i) and (ii).

32. The pharmaceutical combination of claim 29 or claim 30, wherein the combination is formulated for separate administration of (i) and (ii).
